(19) United States
(12) Patent Application Publication
(10) Pub. No.: US 2023/0323301 A1
(43) Pub. Date: Oct. 12, 2023

(54) Protein Tyrosine Phosphatase 1B Inhibited Neutrophils, Neutrophil-Dendritic Cell Hybrids and Uses Thereof

(71) Applicant: WISCONSIN ALUMNI RESEARCH FOUNDATION, Madison, WI (US)

(72) Inventors: Anna Huttenlocher, Madison, WI (US); Igor Slukvin, Verona, WI (US); Morgan Giese, Madison, WI (US); Ho Sun Jung, Madison, WI (US); David Bennin, Mount Horeb, WI (US)

(21) Appl. No.: 18/296,895

(22) Filed: Apr. 6, 2023

Related U.S. Application Data
(60) Provisional application No. 63/327,888, filed on Apr. 6, 2022.
(60) Provisional application No. 63/429,886, filed on Dec. 2, 2022.

(51) Int. Cl. C12N 5/0787; C12N 5/074; C12N 9/22; C12N 9/12; C07K 14/705; C07K 14/74

(52) U.S. Cl. CPC C12N 5/0642; C12N 5/0696; C12N 9/22; C12N 9/12; C07K 14/70532; C07K 14/70539; C12N 5/0031

(57) ABSTRACT

This disclosure provides methods for producing neutrophils under serum-free and feeder-free conditions from protein tyrosine phosphatase 1B (PTP1b)-inhibited pluripotent stem cells. The disclosure further provides PTP1b inhibited neutrophils and uses thereof. Also disclosed are methods for producing human neutrophil-DC hybrid cells and human neutrophil-DC hybrid cells produced thereby.

### CROSS-REFERENCE TO APPLICATION

This application claims priority to U.S. provisional application No. 63/429,886, filed Dec. 2, 2022 and U.S. provisional application No. 63/327,888, filed on Apr. 6, 2022, the disclosures of each application being expression incorporated by reference herein in its entirety.

### STATEMENT REGARDING FEDERALLY SPONSORED RESEARCH

This invention was made with government support under HL142665 and AI134749 awarded by the National Institutes of Health. The government has certain rights in the invention

### STATEMENT REGARDING SEQUENCE LISTING

This application contains a Sequence Listing submitted as an electronic text file named “22-0133-US.xml,” having a size in bytes of 14.2 kb, and created on Apr. 6, 2023. The information contained in this electronic file is hereby incorporated by reference in its entirety.

### FIELD OF THE DISCLOSURE

The disclosure generally relates to methods for producing neutrophils in serum-free and feeder-free conditions from protein tyrosine phosphatase 1B (PTP1b) inhibited pluripotent stem cells. The disclosure further relates to PTP1b inhibited neutrophils and uses thereof.

Also disclosed are methods for producing human neutrophil-dendritic cell (DC) hybrid cells and human neutrophil-DC hybrid cells produced thereby.

### BACKGROUND

Neutrophils are the initial responders to infection and injury and are critical for host survival. Immunocompromised patients are prone to recurrent infections that may require granulocyte transfusion. However, these transfusions have minimal efficacy as donor material is limited and highly heterogeneous. Induced pluripotent stem cells (iPSCs) are a promising option for generating a defined therapeutic that can improve patient outcomes. Current attempts to utilize iPSC-derived neutrophils for this purpose have shown that their function is still limited in comparison to primary human neutrophils. Accordingly, there is a need in the art for efficient and cost-effective protocols for generating iPSC-derived neutrophils.

A subset of neutrophils with antigen presenting capabilities have been identified in patients with autoimmune diseases, such as rheumatoid arthritis or Wegener's granulomatosis (Takashima and Yao, 2014, J. Leuk. Biol. 98: 489-496). These neutrophils have been termed neutrophil-DC hybrids because they express typical dendritic cell (DC) markers CD86+, a T cell costimulatory receptor, and HLADR, the MHC class II receptor. Mouse neutrophil-DC hybrids can also be generated in vitro by stimulation with GM-CSF+/−IL-4 for 2-4 days and display enhanced fungal killing (Fites et al., 2018, PLoS Pathogens 14: e1007073). However, generation of human neutrophil-DC hybrids in vitro has been very challenging due to the short-lived nature of human neutrophils.

Thus there remains a need in the art for efficient and cost-effective protocols for producing neutrophil-DC hybrids that have longer lifetimes than human neutrophils.

### SUMMARY

Provided herein is a method of producing modified neutrophils from pluripotent stem cells, the method comprising:
(a) transiently introducing exogenous ETV2 in human pluripotent stem cells having inhibited expression of protein tyrosine phosphatase 1B (PTP1b) and culturing the ETV2-pluripotent stem cells in serum-free culture medium comprising vascular endothelial growth factor-165 (VEGF-165) and fibroblast growth factor 2 (FGF2), to produce a population of ETV2-hemogenic endothelial cells (ETV2-HECs);
(b) culturing the ETV2-HECs in serum-free and xeno-free culture medium comprising granulocyte-macrophage colony-stimulating factor (GM-CSF) and FGF2 for a sufficient time to produce myeloid progenitors;
(c) culturing the myeloid progenitors in serum-free and xeno-free culture medium comprising granulocyte-colony stimulating factor (G-CSF) and retinoic acid receptor agonist for a time sufficient to differentiate the myeloid progenitors into a population of neutrophils; and
(d) selecting mature neutrophils from the population of neutrophils.

In certain embodiments, the pluripotent stem cells are induced pluripotent stem cells (iPSCs).

In certain embodiments, the culture medium in step (b) further comprises UM171.

As provided herein expression of PTP1b in human pluripotent stem cells is inhibited by gene mutation, RNA-mediated inhibition, RNA editing, DNA gene editing or base editing. In certain embodiments the gene editing methods comprise the use of a nuclease that is a meganuclease, a zinc-finger nuclease, a transcription activator-like effector nuclease (TALEN), or a Cas enzyme. In particular embodiments the nuclease is a Cas enzyme.

In the practice of the methods of this invention the individual steps can be specified wherein step (a) comprises culturing the ETV2-pluripotent stem cells for 1-2 days; step (b) comprises culturing the ETV2-HECs for about 7 to 12 days; and steps (c) and (d) comprise culturing the myeloid progenitor cells for about 6 to 8 days.

In the practice of the methods of this invention cells are selected in step (d) by cell separation, cell sorting, or enrichment methods.

The invention also provides a population of modified mature neutrophils produced by the methods disclosed herein.

In other aspects, the invention provides methods for treating cancer in a patient in need thereof comprising administration of a therapeutically effective amount of a population of modified mature neutrophils provided herein.

In further aspects, the invention provides methods for treating a bacterial infection in a patient in need thereof comprising administration of a therapeutically effective amount of a population of modified mature neutrophils provided herein. In particular embodiments the bacterial infection is a systemic infection.

The invention further provides pluripotent stem cells having inhibited expression of protein tyrosine kinase phosphatase 1B (PTP1b). In certain embodiments, PTP1b expression in the pluripotent stem cells provided by the invention are produced by gene mutation, RNA-mediated inhibition, RNA editing, DNA gene editing or base editing. In certain embodiments the gene editing methods comprise the use of a nuclease that is a meganuclease, a zinc-finger nuclease, a transcription activator-like effector nuclease (TALEN), or a Cas enzyme. In particular embodiments the nuclease is a Cas enzyme.

Also provided herein are methods for producing human neutrophil-dendritic cell (DC) hybrids in vitro comprising the steps of:
(a) transiently introducing exogenous ETV2 in human pluripotent stem cells having inhibited expression of protein tyrosine phosphatase 1B (PTP1b) and culturing the ETV2-pluripotent stem cells in serum-free culture medium comprising vascular endothelial growth factor-165 (VEGF-165) and fibroblast growth factor 2 (FGF2), to produce a population of ETV2-hemogenic endothelial cells (ETV2-HECs);
(b) culturing the ETV2-HECs in serum-free and xeno-free culture medium comprising granulocyte-macrophage colony-stimulating factor (GM-CSF) and FGF2 for a sufficient time to produce myeloid progenitors;
(c) culturing the myeloid progenitors in serum-free and xeno-free culture medium comprising granulocyte-colony stimulating factor (G-CSF) and retinoic acid receptor agonist for a time sufficient to differentiate the myeloid progenitors into a population of neutrophils;
(d) harvesting the neutrophil population and resuspending the cells in serum-supplemented or serum-free culture media with comprising GM-CSF for an additional two days to produce human neutrophil-DC hybrids; and
(e) selecting human neutrophil-DC hybrids from the culture.

In certain embodiments, the pluripotent stem cells used in this aspect of the invention are induced pluripotent stem cells (iPSCs).

In certain embodiments, the culture medium in step (b) further comprises UM171.

In the practice of these methods of this invention the individual steps can be specified wherein step (a) comprises culturing the ETV2-pluripotent stem cells for 1-2 days; step (b) comprises culturing the ETV2-HECs for about 7 to 12 days; and step (c) comprises culturing the myeloid progenitor cells for about 6 to 8 days, and step (d) comprises culturing the cells for an additional 2 days before selecting human neutrophil-DC hybrids from the culture in step (e).

In the practice of the methods of this invention cells are selected in step (e) by cell separation, cell sorting, or enrichment methods.

The invention also provides a population of human neutrophil-DC hybrids produced by the methods disclosed herein.

In other aspects, the invention provides methods for treating cancer in a patient in need thereof comprising administration of a therapeutically effective amount of a population of human neutrophil-DC hybrids provided herein.

In further aspects, the invention provides methods for treating a bacterial infection in a patient in need thereof comprising administration of a therapeutically effective amount of a population of human neutrophil-DC hybrids provided herein. In particular embodiments the bacterial infection is a systemic infection.

These and other features, objects, and advantages of the present invention will become better understood from the description that follows. In the description, reference is made to the accompanying drawings, which form a part hereof and in which there is shown by way of illustration, not limitation, embodiments of the invention. The description of certain specific embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

FIG. 1A-FIG. 1E shows the generation of PTP1B KO iPSC-derived neutrophils. FIG. 1A is a diagram illustrating sgRNAs targeting of exon 3 of PTPN1 for CRISPR/Cas9 mediated deletion of a 67 bp region at the stem cell stage, and a timeline for neutrophil differentiation from bone marrow derived iPSCs. FIG. 1B is a western blot confirming PTP1B CRISPR/Cas9 mediated deletion in differentiated neutrophils. FIG. 1C are photomicrographs of representative cytospins showing morphological confirmation of neutrophil differentiation. FIG. 1D is a histogram of cells surface receptor expression data from flow cytometry staining of differentiated neutrophils and myeloid cells, wherein cells were gated on live cells then percent quantified of CD11b+. FIG. 1E shows cell viability of iPSC-derived neutrophils compared to human peripheral blood (PB) neutrophils. Means±SEM are shown.

FIG. 2A-FIG. 2E shows that deletion of PTP1B promotes intracellular signaling and neutrophil motility. FIG. 2A is a schematic diagram of a LD microfluidic device used to live image neutrophil migration. FIG. 2B shows representative western blots and quantification of ERK, HS1, AKT phospho signaling after stimulation of PTP1B KO iPSC-derived neutrophils with 1 uM FMLP for 3 minutes. FIG. 2C is a chemotactic index and mean velocity for iPSC-derived neutrophils (termed “iNeutrophils” herein) in response to an FMLP gradient. FIG. 2D shows representative track plots of cells migrating in response to FMLP gradient. Blue tracks indicate cells that traveled towards the FMLP source, whereas red tracks indicate cells that moved away. FIG. 2E shows representative immunofluorescence images of F-actin staining after 100 nM FMLP stim, and a bar graph showing quantified integrated density of F-actin staining. Means±SEM are shown.

FIG. 3A-FIG. 3D shows the effect of PTP1B deletion on bulk iNeutrophil function. FIG. 3A shows iNeutrophil phagocytosis of acidified pHrodo E. coli beads quantified by flow cytometry. Cells were gated on CD11b+ and the percent of pHrodo+ cells were quantified. FIG. 3B is a graph showing quantification of iNeutrophil intracellular reactive oxygen species (ROS) production over time using DHR123 peroxynitrite indicator following stimulation with 50 ng/mL phorbol 12-myristate 13-acetate (PMA). FIG. 3C is a graph showing quantification of iNeutrophil and human peripheral blood neutrophil intracellular ROS production over time using DHR123 peroxynitrite indicator following stimulation with 50 ng/mL PMA. FIG. 3D is a bar graph showing NETosis quantified with Sytox Green DNA indicator after 4-hour stimulation with 100 ng/mL PMA. Means±SEM are shown.

FIG. 4A-FIG. 4D shows deletion of PTP1B increases IL-8 inflammatory cytokine production. Bar graphs show results of quantitative polymerase chem reaction (qPCR) analysis of inflammatory cytokines IL1B (FIG. 4A), IL6 (FIG. 4B), CXCL8 (FIG. 4C), and TNF (FIG. 4D) after 2-hour stimulation with 200 ng/mL E. coli LPS. FIG. 4E is a bar graph showing quantification of IL-8 protein by ELISA after stimulation with 200 ng/mL LPS or 10 ug/mL Zymosan for 4 hours. Samples were normalized to the WT unstimulated control. Means±SEM are shown.

FIG. 5A is a bar graph showing Candida auris fungal survival (detected by staining with Presto Blue) after 4 hours co-incubation. FIG. 5B is a bar graph showing that PTP1b KO neutrophils isolated using CD15+ bead selection for mature neutrophils had similar neutrophil marker expression compared to wild-type neutrophils. FIG. 5C is a bar graph showing that when gated on CD15+ or CD15+CD16+ neutrophils by flow cytometry staining, PTP1B KO neutrophils had improved phagocytosis over wild-type neutrophils. FIG. 5D is a bar graph showing that PTP1b KO neutrophils after CD15+ bead selection for mature neutrophils had fungal killing comparable to wild-type neutrophils and primary human neutrophils.

FIG. 6A-FIG. 6D illustrates that PTP1B KO neutrophils showed increased activation and swarming in response to A. fumigatus. FIG. 6A shows representative bright-field images of A. fumigatus expressing RFP co-cultured with WT or PTP1B-KO iNeutrophils over the course of 8 hours. FIG. 6B shows higher magnification bright-field images of WT or PTP1B-KO iNeutrophils cell morphology and interaction with A. fumigatus hyphae. FIG. 6C is bar graph showing quantification of percent of A. fumigatus germlings surrounded by iNeutrophils. FIG. 6D is a bar graph showing quantification of fungal killing after co-culture with WT or PTP1B-KO iNeutrophils after 4 hours. Experiments were conducted at least three times, or as indicated on the plot.

FIG. 7A-FIG. 7D shows iNeutrophil differentiation with GM-CSF treatment. FIG. 7A is schematic diagram of a timeline for neutrophil-DC hybrid differentiation from bone marrow derived iPSCs. FIG. 7B is a graph showing cell viability of iPSC-derived neutrophils and neutrophil-DC hybrids compared to human peripheral blood (PB) neutrophils. FIG. 7C are representative cytospins showing morphological confirmation of neutrophil differentiation. FIG. 7D is a bar graph showing flow cytometry staining of differentiated neutrophils and myeloid cells with and without 10 ng/mL GM-CSF treatment. Cells were gated on Live cells then percent quantified of CD11b+. Means±SEM are shown.

FIG. 8A-FIG. 8B shows GM-CSF treatment of iNeutrophils induces expression of antigen presenting markers. FIG. 8A is a graph showing histograms of normalized expression of cell surface receptor staining by flow cytometry across samples. FIG. 8B shows single cell clustering based upon cell surface receptor staining of flow cytometry samples. Expression values are scaled from 0 to 1, after restricting the data to the 1st and 99th percentiles.

FIG. 9A-FIG. 9C shows PTP1B knockout increases differentiation of neutrophil-DC hybrids via JAK2 signaling. FIG. 9A shows a gating strategy to identify neutrophil-DC hybrid population by flow cytometry. FIG. 9B is a bar graph showing quantification of neutrophil-DC hybrids differentiated with or without 10 ng/mL GM-CSF treatment. FIG. 9C is a representative western blot staining of phospho JAK2 after stimulation with 100 ng/mL GM-CSF at 0, 5, 10, and 20 minutes, accompanied by a graph showing quantified western blot staining of pJAK2 normalized to total JAK2 at 4 independent time points (0, 5, 10, or 20 minutes). Means±SEM are shown.

FIG. 10A-FIG. 10B shows neutrophil-DC hybrids have enhanced phagocytosis. FIG. 10A is a bar graph showing iNeutrophil phagocytosis of acidified Phrodo E. coli beads quantified by flow cytometry. Percent of Phrodo+ neutrophils gated on CD11b+ CD15+ CD16+. FIG. 10B is a bar graph showing percent of Phrodo+ Neutrophil-DC hybrids gated on CD11b+ CD14− CD15+ CD16+ HLADR+ CD86+. Means±SEM are shown.

FIG. 11A-FIG. 11D shows PTP1B-null neutrophils have increased expression of granule markers. FIG. 11A illustrates flow cytometry data showing the percent of MPO+ cells gated on CD11b+ myeloid cells with and without GM-CSF treatment. FIG. 11B is a representative contour plot of MPOhi CD11b+ CD15+ CD16+ neutrophils quantified by flow cytometry. FIG. 11C is a bar graph showing quantification of MPOhi iNeutrophils with and without GM-CSF treatment. FIG. 11D is a bar graph showing quantification of CD66b+ iNeutrophils with and without GM-CSF treatment. Means±SEM are shown.

FIG. 12A-FIG. 12B show GM-CSF treatment improves neutrophil control of A. fumigatus. FIG. 12A are representative bright-field images of A. fumigatus expressing RFP co-culture with WT or PTP1B-KO iNeutrophils over the course of 8 hours. iNeutrophils were untreated or cultured in the presence of 10 ng/mL GM-CSF for 2 days. FIG. 12B shows higher magnification bright-field images of GM-CSF treated WT or PTP1B-KO iNeutrophils cell morphology and interaction with A. fumigatus hyphae.

FIG. 13 is a bar graph comparing A. fumigatus killing by GM-CSF treated PTP1B KO hybrids compared to WT cells.

### DETAILED DESCRIPTION

The disclosure generally provides methods for producing neutrophils under serum-free and feeder-free conditions from protein tyrosine phosphatase 1B (PTP1b) inhibited pluripotent stem cells. The disclosure further relates to such PTP1b inhibited neutrophils and uses thereof. The disclosure also provides methods for producing PTP1b inhibited neutrophil-dendritic cell (DC) hybrids under serum-free and feeder-free conditions from protein tyrosine phosphatase 1B (PTP1b) inhibited pluripotent stem cells, and such PTP1b neutrophil-C hybrid and uses thereof.

All publications, including but not limited to patents and patent applications, cited in this specification are herein incorporated by reference as though set forth in their entirety in the present application.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

“Pluripotent stem cells” refer to cells having the capacity to differentiate into cells of all three germ layers. “Embryonic stem cells” or “ESCs” mean a pluripotent cell or population of pluripotent cells derived from an inner cell mass of a blastocyst. See Thomson et al., (1998) Science 282:1145-1147.

“Induced pluripotent stem cells” or “iPS cells” refer to a pluripotent cell or population of pluripotent cells that may vary with respect to their differentiated somatic cell of origin, that may vary with respect to a specific set of potency-determining factors and that may vary with respect to culture conditions used to isolate them, but nonetheless are substantially genetically identical to their respective differentiated somatic cell of origin and display characteristics similar to higher potency cells, such as ESCs. See, e.g., Yu et al., (2007) Science 318:1917-1920.

“Hemogenic endothelial cells (HECs)” refer to a subset of endothelial cells that can differentiate into hematopoietic cells.

“Myeloid progenitors” are cells capable of differentiating into cell types of the myeloid lineages.

“Chemically-defined culture” “fully defined, growth factor free culture conditions,” and “fully-defined conditions” indicate that the identity and quantity of each medium ingredient is known and the identity and quantity of supportive surface is known.

“Xeno-free culture medium” refers to medium that does not contain any components derived from animal sources, such as, for example, serum.

Uses of Neutrophils Using ETV2 Modified mRNA Differentiation Systems

The present disclosure provides methods for efficient neutrophil production from pluripotent stem cells using direct programming with transient expression of ETV2, e.g., by addition of modified mRNA (mmRNA) of ETV2 into the human iPSCs (hiPSCs). Initially, hiPSCs were directly programmed into hemogenic endothelial cells using ETV2 mmRNA which transiently produced ETV2 within the cells. Next, the hemogenic endothelial cells were then differentiated into myeloid progenitors in the presence of GM-CSF, FGF2 and optionally UM171 (the presence of UM171 in combination with GM-CSF and FGF2 increases the number of neutrophils produced by the methods). Myeloid progenitors which are non-adherent could be continuously collected from cultures every 8-10 days for up to 30 days of post ETV2 transfection. Finally, these myeloid progenitors were subsequently differentiated into neutrophils. The methods for neutrophil production from pluripotent stem cells using direct programming with transient expression of ETV2 are described in U.S. Publication No. 20200385676, the contents of which are incorporated by reference in its entirety.

Provided herein are methods for producing neutrophils having inhibited expression of protein tyrosine phosphatase 1B (PTP1b), such methods comprising:
(a) transiently introducing exogenous ETV2 in human pluripotent stem cells having inhibited expression of protein tyrosine phosphatase 1B (PTP1b) and culturing the ETV2-pluripotent stem cells in serum-free culture medium comprising Vascular endothelial growth factor-165 (VEGF-165) and fibroblast growth factor 2 (FGF2), to produce a population of ETV2-hemogenic endothelial cells (ETV2-HECs);
(b) culturing the ETV2-HECs in serum-free and xeno-free culture medium comprising granulocyte-macrophage colony-stimulating factor (GM-CSF) and FGF2 for a sufficient time to produce myeloid progenitors;
(c) culturing the myeloid progenitors in serum-free and xeno-free culture medium comprising granulocyte-colony stimulating factor (G-CSF) and retinoic acid receptor agonist for a time sufficient to differentiate the myeloid progenitors into a population of neutrophils; and
(d) selecting mature neutrophils from the population of neutrophils.

The pluripotent stem cells used in the methods disclosed herein have inhibited expression of protein tyrosine phosphatase 1B (PTP1b). The term “having inhibited expression of PTP1b,” indicates that expression of the PTP1b gene is repressed or the protein encoded therein is not expressed in a functional protein form. In particular embodiments, expression of PTP1b is knocked out so that that there is no detectable expression of PTP1b. This inhibition or knockout can be obtained by gene mutation, RNA-mediated inhibition, RNA editing, DNA gene editing or base editing.

In particular embodiments, the gene editing method comprises the use of a nuclease selected from a meganuclease, zinc-finger nucleases (ZFNs), transcription activator-like effector nucleases (TALENs), and Cas enzyme. In particular embodiments, the nuclease is a Cas9 enzyme.

ETV2 can be transiently introduced into the PSCs by methods known in the art. Methods of transiently expressing ETV2 in PSCs include, but are not limited to, for example, introducing transiently exogenous nucleic acids encoding the protein of interest (e.g., by plasmid expression vector transfection, or modified mRNA transfection) or protein transduction, among others. In one embodiment, mmRNA of ETV-2 (e.g., Accession No: NM_014209.2; SEQ ID NO:15) is introduced into the PSCs by suitable methods. Methods of transiently expressing ETV2 in PSCs are described in U.S. Pat. No. 9,382,531, the contents of which are incorporated by reference in its entirety. Methods of introducing mmRNA into PSCs are known in the art, and include, but are not limited to, the method described in the Examples, for example, by transfection or electroporation. The methods of introducing mmRNA or DNA to transiently express ETV-2 protein is within the skill of one in the art and are not limited to what is demonstrated in the Examples herein.

After initiating transient expression of ETV2 in the PSCs, these cells are cultured for a sufficient time to produce a population of ETV2-hemogenic endothelial cells (ETV2-HECs). In particular embodiments, a sufficient time is a period of about 24 hours to about 4 days. In some embodiments, a sufficient amount of time to produce a population of ETV2-hemogenic endothelial cells comprises culturing the ETV2-cells for about 1-2 days. In some embodiments, a sufficient amount of time to produce a population of ETV2-hemogenic endothelial cells comprises culturing the ETV2-cells for about 3-8 days, for example, for about 4 days. For example, in some embodiments, the step to produce a population of ETV2-hemogenic endothelial cells comprises culturing for 3 days, alternatively 4 days, alternatively 5 days, alternatively 6 days, alternatively 7 days, alternatively 8 days to produce ETV2-hemogenic endothelial cells.

In some embodiments, the culture medium is “serum-free” which refers to cell culture materials that are free of serum obtained from animal or human (e.g., fetal bovine) blood.

In some embodiments, the culture conditions are feeder-free, meaning that the conditions do not use feeder cells. In particular embodiments, the culture conditions are serum-free and feeder-free.

In particular embodiments, the methods disclosed herein use a maintenance culture medium for culturing the PSCs after transfection with ETV2 mmRNA. In particular embodiments the culture medium is E8-TeSR. E8 culture medium” and “E8” are used interchangeably and refer to the chemically defined culture medium having the following defined components: DMEM/F12, L-ascorbic acid-2-phosphate magnesium, sodium selenium, and NaHCO₃ in a final volume of 200, transferrin, insulin, FGF2 and TGFβ1.

In some embodiments, the culture medium is a xeno-free cell culture medium. In some embodiments, the culture condition is xeno-free, serum free and feeder-free.

Serum-free and xeno-free medium suitable for culturing the ETV2-hemogenic endothelial cells to produce myeloid progenitors are known in the art and include, but are not limited to, for example, StemLine II (commercially available from Sigma Aldrich).

In some embodiments, a sufficient amount of time for culturing the ETV2-HECs to produce myeloid progenitors is at least 4 days, for example, at least 4-23 days. In some embodiments, a sufficient amount of time for culturing the ETV2-HECs to myeloid progenitors is about 7 to 12 days.

In some embodiments, the methods disclosed herein comprise isolating the myeloid cells from the culture. Suitable methods of isolating the cells are known in the art. In one example, non-adherent cells can be collected from the culture leaving the adherent cells behind. In some embodiments, the adherent cells isolated from the non-adherent myeloid cells, may be used in methods of producing neutrophils.

For production of neutrophils, the methods comprise the step of culturing the myeloid progenitors in a culture medium comprising granulocyte-colony stimulating factor (G-CSF) and a retinoic acid receptor agonist for a sufficient time to differentiate the myeloid progenitors into modified neutrophils. In some embodiments the retinoic acid receptor agonist is AM580 (a retinoic acid receptor agonist (Stem Cell #72964) having a structure identified as CAS No. 102121-60-8.

A suitable time to differentiate the myeloid progenitors into modified neutrophils includes for at least 9 days, for example at least 9-21 days. In some embodiments, a suitable time for culturing the myeloid progenitors to differentiate into modified neutrophils is about 6 to 8 days.

Serum-free and xeno-free medium suitable for culturing the myeloid progenitors to differentiate into modified neutrophils are known in the art and include, but are not limited to, for example, StemSpan™ H3000 (StemCell Technologies).

In particular embodiments, the methods disclosed herein comprise selecting mature neutrophils from the population of neutrophils. In some embodiments, the mature neutrophils are selected using a cell separation, cell sorting, or enrichment method, e.g., fluorescence activated cell sorting (FACS), enzyme-linked immunosorbent assay (ELISA), magnetic beads, magnetic activated cell sorting (MACS), and combinations thereof.

In particular embodiments, a mature neutrophil is identified by expression of cell surface markers. In particular embodiments, a mature neutrophil expresses CD15 and/or CD16.

Also provided herein is a population of modified mature neutrophils produced by the methods disclosed herein. The modified mature neutrophils have inhibited expression of PTP1b as well as superior anti-microbial and phagocytosis for therapeutic purposes.

In certain embodiments, provided herein are neutrophil-dendritic cell (DC) hybrid cells. These cells are produced as set forth herein by further incubating neutrophils for an additional two days in the presence of an effective amount of GM-CSF (granulocyte macrophage colony stimulating factor). Advantageously, the cells are cultured in media containing 10 ng/mL GM-CSF.

While the methods disclosed herein include ordered, sequential events, the timing of the events may be varied by at least 20%. For example, while a particular step may be disclosed in one embodiment as lasting one day, the event may last for more or less than one day. For example, “one day” may include a period of about 18 to about 30 hours. Periods of time indicated that are multiple day periods may be multiples of “one day,” such as, for example, two days may span a period of about 36 to about 60 hours, and the like. In another embodiment, time variation may be lessened, for example, where day 2 is 48+/−3 hours from d0; day 4 is 96+/−3 hours from d0, and day 5 is 120 hours+/−3 hours from d0.

### Methods For Use of the Modified Neutrophils and Neutrophil-Dendritic Cell (DC) Hybrids

In particular embodiments, PTP1b knockout neutrophils or neutrophil-DC hybrids disclosed herein are useful for treating or preventing various disorders such as a cancer or an infection. In particular embodiments provided herein is a method of treating cancer comprising administering the PTP1b knockout neutrophils disclosed herein. In particular embodiments provided herein is a method of treating an infection comprising administering the PTP1b knockout neutrophils disclosed herein. In some embodiments, the infection is a systematic infection.

The terms “treatment” or “treat,” as used herein, refer to both therapeutic treatment and prophylactic or preventative measures. Those in need of treatment include subjects having cancer as well as those prone to having cancer or those in cancer is to be prevented. In some embodiments, the methods, compositions, and combinations disclosed herein can be used for the treatment of cancer. In other embodiments, those in need of treatment include subjects having an infection as well as those prone to have an infection or those in which an infection is to be prevented. In certain embodiments, the methods, compositions, and combinations disclosed herein can be used for the treatment of infections.

In some cases, neutrophils or neutrophil-DC hybrids obtained according to a method provided herein can be administered as a pharmaceutical composition comprising a therapeutically effective amount of neutrophils as a therapeutic agent (i.e., for therapeutic applications).

The terms “pharmaceutical composition” or “therapeutic composition,” as used herein, refer to a compound or composition capable of inducing a desired therapeutic effect when properly administered to a subject. In some embodiments, the disclosure provides a pharmaceutical composition comprising a pharmaceutically acceptable carrier and a therapeutically effective amount of the neutrophils of the disclosure.

The terms “pharmaceutically acceptable carrier” or “physiologically acceptable carrier,” as used herein, refer to one or more formulation materials suitable for accomplishing or enhancing the delivery of the neutrophils of the disclosure.

The term “subject” is intended to include human and non-human animals, particularly mammals. In certain embodiments, the subject is a human patient.

The terms “administration” or “administering,” as used herein, refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

Without limiting the disclosure, a number of embodiments of the disclosure are described below for purpose of illustration.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Generation of PTP1b Knockout Neutrophils and Characterization Thereof

### Materials and Methods

### Cell Culture

Wild type bone marrow-derived IISH2i-BM9 (Yu et al., 2009, Science 324:797-801) hiPSCs were obtained from WiCell (Madison, WI). Human induced pluripotent stem cells (hiPSCs) with knockout PTP1b gene were generated using CRISPR/Cas9 technology. Specifically, to generate PTP1B knockout BM9 iPSCs, two single guide RNAs were designed using CRISPR design tool (Synthego). Two sgRNA sequences are

GATGTAGTTTAATCCGACTA (sgRNA1; SEQ ID NO. 11) and
TAAAAATGGAAGAAGCCCAA (sgRNA2; SEQ ID NO. 12). BM9 iPSCs were electroporated with 5 μg each of the two sgRNAs and 5 μg Cas9 protein (PNA Bio), and then plated at a low density in 6 well plate. After 7 days, individual colonies were picked and further expanded.
After expansion, individual clones were screened by genomic PCR (primer1:
TGCATCAGAGAACAGATCCT (SEQ ID NO. 13) and primer2:
CTGGGTAAGAATGTAACTCC) (SEQ ID NO. 14) for the acquisition of 67 bp deletion in wildtype BM9iPSCs. Genomic PCR with these primers show a 461 bp product from wild type BMP iPSCs and a 394 bp product from PTP1B knockout iPSCs.

Wild type and PTP1b^{-/-} hiPSCs were cultured on Matrigel-coated tissue culture plates in E8 medium (STEMCELL Technologies) (Chen et al., 2011, Nat Methods. 8:424-9).

### Stem Cell Culture and Neutrophil Differentiation

Neutrophils were differentiated from bone marrow derived hiPSCs as previously described (Majumder et al, 2020, STAR Protoc. 1: 100075). Briefly, bone marrow-derived IISH2i-BM9 (Yu et al., 2009, Id.) were obtained from WiCell (Madison, WI). hiPSCs were cultured on Matrigel-coated tissue culture plates in E8 medium (STEMCELL Technologies, Vancouver, Canada) (Chen et al., 2011, Id.).

To induce hemogenic endothelium, hiPSCs were transfected with ETV2 mmRNA in mTeSR™-E8™ complete media using TransIT reagent and mRNA boost. One hour prior to transfection, cells were detached by TrypLE Select (LifeTech) to singularize. Cells were re-plated onto collagen (2.4 ug/ml) coated plates in E8 with 10 uM ROCK inhibitor (ROCKi; Tocris Y-27632) for transfection according to the manufacturer's protocol. One day following transfection, media was changed to StemLineII media with 20 ng/mL VEGF-165 and 10 ng/mL FGF to induce differentiation into hemogenic endothelial cells. This media cocktail is designated Media A herein. After two days, the media was changed to differentiate the cells into common myeloid progenitors (CMPs) with StemLineII media supplemented with FGF2 (20 ng/mL), GM-CSF (25 ng/mL) (PeproTech), and UM171 (50 nM; Xcess Biosciences). This media cocktail is designated Media B herein. On days 8-10, floating cells were gently harvested and used for terminal neutrophil differentiation. These cells were cultured in StemSpan SFEM II medium (STEMCELL Technologies), supplemented with GlutaMAX 100× (Thermo Fisher Scientific), ExCyte 0.2% (Merck Millipore), human G-CSF (150 ng/mL; Amgen), and AM580 retinoic acid agonist 2.5 μM (Sigma-Aldrich) at 2×10⁶ cells/mL density. This media cocktail is designated Media C herein. After 4 days, fresh Media C was added on the top of the cells. Neutrophils were harvested from the supernatant 8-10 days after plating in Media C.

### Production of Neutrophil-DC Hybrids

To differentiate iPSC-derived neutrophils into neutrophil-DC hybrids, a final step was added to the culture protocol. Cells were harvested from Media C after 6-8 days and then resuspended in Media C supplemented with 10% FBS and GM-CSF (10 ng/mL) (PeproTech). The cells were cultured for an additional two days and then harvested for further use.

### Generation of PTP1B^{-/-} BM9iPSCs

To generate PTP1B knockout BM9 iPSCs, two single guide RNAs were designed in CRISPR design tool (Synthego) and genetic modification performed as disclosed above. Prior to nucleofection, BM9 iPSCs were treated with 10 μM ROCKi, detached by TrypLE Select (LifeTech), and singularized by pipetting. 5 ug of both sgRNAs and 5 ug of Cas9 protein (PNA Bio) were incubated together for 10 minutes, then the cells were nucleofected using the Human Stem Cell Nucleofector Kit 2 (Lonza, #VPH-5022). Cells were plated at a low density on a Matrigel coated 10 cm plate in mTeSR plus+1× CloneR supplement (StemCell #05888). After 2 days, media was changed to mTeSR plus media alone. Individual colonies were picked after 7 days and further expanded. To confirm biallelic mutation the PTP1B gene, genomic DNA was extracted from individual clones, then screened by PCR for the acquisition of a 67 bp deletion in the wild-type PTP1B allele using primers P1 and P2 (SEQ ID NOs. 13 and 14, respectively). After verification of multiple PTP1B−/− clones, a single clone (#6) was selected for further experimentation.

### Human Neutrophil Isolation

Human blood was obtained from volunteering donors with informed written consent through a protocol that was approved by the Internal Review Board of the University of Wisconsin-Madison. Neutrophils were isolated using MACSxpress negative antibody selection kit and purified with the MACSxpress erythrocyte depletion kit (Miltenyi Biotec, Inc., Auburn, CA), following manufacturer's instructions. Isolated neutrophils were resuspended in modified HBSS (+0.1% HSA+10 mM Hepes) and utilized as disclosed herein. Incubations involving neutrophils were performed at 37° C. with 5% CO₂.

### Cytospins

To confirm neutrophil morphology, 90,000 cells were spun onto a glass slide for 5 minutes at 1200 rpm using a Shandon Cytospin 3 centrifuge (ThermoFisher). Cytospin slides were then stained with Differential Quick III Stain Kit (Electron Microscopy Sciences cat#26096-25) following manufacturer's instructions. Slides were imaged at 20× on an Olympus IX70 microscope and evaluated for the presence of hypersegmented nuclei.

### Western Blot Cell Signaling

Western blotting was conducted to evaluate phospho-signaling in these iPSC-derived neutrophils. Cells were stimulated with 1 uM FMLP for 3 minutes. Cell pellets were collected in lysis buffer (25 mM HEPES, pH 7.5, 150 mM NaCl, 1% Nonidet P-40, 10 mM MgCl₂, 1 mM EDTA, 10% glycerol supplemented with 1 g/ml pepstatin A, 2 g/ml aprotinin, 1 g/ml leupeptin, and 200 nM phenylmethanesulfonyl fluoride (PMSF), 1 mM sodium orthovanadate, and 1% protease inhibitor mixture 2 (Sigma #p5726)). For GM-CSF stimulation, cells were incubated with 100 ng/mL GM-CSF for 5, 10, or 20 minutes. Pellets were collected in Triton lysis buffer (50 mM Tris-HCL, 50 mM NaCl, 5 mM EDTA, 1% Triton and 2× Halt Protease and Phosphatase inhibitors (Thermo Fisher #78440). For both cell stimulations, cells were incubated on ice for 10 minutes and then sonicated with 20% amplitude for 3× 5 sec. Cells were then clarified by centrifugation at 15,000×g, 4° C. for 15 min. Protein concentrations were determined using the Pierce BCA Protein Assay (Thermo Scientific; 23225) and samples stored at −80° C. Immunoblotting of cell lysates was performed and blots were imaged with an infrared imaging system (Odyssey; LI-COR Biosciences). Primary and secondary antibodies used can be found in Table 1.

TABLE 1
Immunoblot antibodies utilized herein

| Target | Vendor | Catolog No. |
|---|---|---|
| Rabbit Anti-Human AKT | Cell Signaling | 9272S |
| Mouse Anti-Human phopsho AKT | Cell Signaling | 4051 |
| Rabbit Anti-Human HS1 (D83A8) | Cell Signaling | 3890S |
| Rabbit Anti-Human phospho HS1 | Cell Signaling | 4507S |
| Y397 | | |
| Rabbit Anti-human ERK | Thermo Fisher | 44-654G |
| Mouse Anti-Human phospho ERK | Abcam | ab50011 |
| Rabbit Anti-Human JAK2 | Thermo Fisher | AHO1352 |
| Mouse Anti-Human phospho JAK2 | Abcam | ab32101 |
| Goat anti-mouse IgG (H&L) | Rockland | 610-140-002-0.5 |
| Antibody Dylight 800 Conjugated | Immunochemicals | |
| Goat anti-Rabbit IgG (H&L) | Invitrogen | A-21076 |
| Antibody Cross-Adsorbed | | |
| Secondary Antibody, 680 | | |
| Conjugated | | |

### Immunofluorescent Imaging

Acid-washed 22-mm glass circle coverslips were coated with 10 μg/ml fibronectin for at least 1 h at 37° C. and then blocked for 30 min with 2% BSA-PBS. Cells (3×10⁵) in 500 μl 0.5% HSA-RPMI were seeded per coverslip in a 24-well plate (one coverslip per well) and allowed to rest for 30 min. FMLP was added for a final concentration of 100 nM fMLP and cells were allowed to adhere for 30 minutes at 37° C., 5% CO₂. Media was aspirated and fixation was performed as follows: 1 ml of 37° C. preheated 4% paraformaldehyde in PEM buffer (80 mM PIPTES, pH6.8; 5 mM EGTA, pH 8.0; 2 mM MgCl₂) for 15 min at RT. This was aspirated and 0.25% Triton X-100 in PEM buffer was added for 10 min at 37° C. Coverslips were washed three times with PBS, blocked in 5% BSA-PBS for 60 min at RT, washed, and incubated in Rhodamine-phalloidin (Thermo Fisher Cat#R415) overnight at 4° C. Coverslips were washed and incubated in secondary antibody solution for 60 min at RT. Coverslips were washed, counterstained with Hoechst 33342 (Thermo Fisher H3570; 1:500) for 5-10 min, washed with doubly-distilled water (ddH₂O), and mounted on Rite-On Frosted Slides (Fisher Scientific; 3050-002) with ProLong Gold Antifade Mountant (Invitrogen; P36930).

### CD15 Positive Selection

hiPSC-derived neutrophils were positively selected for mature neutrophil markers. CD15 microbeads were incubated with neutrophils following the manufacturer's protocol (Miltenyi Cat#130-046-601) and then positively selected using LD columns (Miltenyi Cat#130-042-901). Cells were allowed to rest overnight in Media C before use in further experiments.

For selection of CD15 positive neutrophil-DC hybrids, cells were selected and then cultured in Media C with added 10% FBS and 10 ng/mL GM-CSF for two days.

### Flow Cytometry

Flow cytometry analysis was used to evaluate expression of cell lineage markers on iPSC-derived neutrophils with and without GM-CSF treatment. Neutrophils were stained in PBS+1% HSA media+Brilliant Buffer (Thermo Fisher #00-4409-42) and Human TruStain FcX Fc Receptor Blocking Solution (Biolegend #422302), then fixed with 2% PFA. Data acquisition and analysis were performed on an Aurora Cytometer (CytekBio). Antibodies used in this study can be found in Table 2. Live cells were identified with Ghost Dye Red 780 or Zombie NIR dye. Myeloid cells were identified by CD11b+ expression and neutrophils were identified by CD11b+ and CD15+ or CD16+ expression. Monocytes were identified as CD14+ cells. Neutrophil-DC hybrids were identified as CD11b+CD15+CD16+HLADR+CD86+.

TABLE 2
Flow Cytometry antibodies and dyes used herein

| Target | Fluor | Clone | Cat # | Vendor |
|---|---|---|---|---|
| CD14 | Spark Blue | 63D3 | 367148 | Biolegend |
| | 550 | | | |
| CD71 | PE/Dazzle | CY1G4 | 334120 | Biolegend |
| CD86 | BV650 | IT2.2 | 305428 | Biolegend |
| CD117 | BV711 | 104D2 | 313230 | Biolegend |
| CD16 | AF700 | 3G8 | 302026 | Biolegend |
| CD66b | PE/Fire640 | 6/40c | 392918 | Biolegend |
| CD182 | BV605 | 5A12 | 743421 | FisherScientific |
| (CXCR1) | | | | |
| CD182 | BV605 | 5A12 | 744197 | FisherScientific |
| (CXCR2) | | | | |
| CXCR4 | APC | 12G5 | 306510 | Biolegend |
| CD10 | PE | HI10a | 312203 | Biolegend |
| CD11b | PECy7 | ICRF44 | 301321 | Biolegend |
| CD15 | APC-Fire 810 | W6D3 | 323058 | Biolegend |
| HLA-DR | BUV805 | L203 | 752497 | BD Biosciences |
| Zombie NIR | 746 | | 423105 | FisherScientific |
| CD11b+ | PE | M1/70 | 101208 | Biolegend |
| CD15 | APC | W6D3 | 323008 | Biolegend |
| CD16 | BV711 | 3G8 | 563127 | BD Biosciences |
| CD14 | BUV805 | M5E2 | 612902 | BD Biosciences |
| HLADR | PE-Cy7 | L243 | 307616 | Biolegend |
| CD86 | AF700 | FUN-1 | 561124 | BD Biosciences |
| MPO | FITC | MPO455-8E6 | 11-1299-42 | Thermo Fisher |
| Ghost Dye | 780 | | 13-0865-T100 | Tonbio |
| Red | | | | Biosciences |
| Phrodo Green | 509/533 | | P35373 | Thermo Fisher |
| AM | | | | |
| Intracellular | | | | |
| pH Indicator | | | | |
| pHrodo | 509/533 | | P35366 | Thermo Fisher |
| Green E. coli | | | | |
| Bioparticles | | | | |
| Brilliant | | | 00-4409-42 | Thermo Fisher |
| Buffer | | | | |
| Human TruStain FcX | | | 422302 | Biolegend |
| Fc Receptor | | | | |
| Blocking | | | | |
| Solution | | | | |
| Ultracomp | | | 501129040 | Thermo Fisher |
| ebeads | | | | |

### Flow Analysis

Unmixed flow files were pre-gated on live (Zombie negative) single cells in flowjo (v10). FCS files were exported for each sample and later read into R (v4.0) for further analysis. Flow files were used to generate a “flowset” object (CytofWorkflow, v.1.14.0), which stored expression values for each marker and associated experiment metadata (Nowicka et al, 2017, F1000res. 6: 748). Expression data were normalized using the recommended arcsinh transformation and a cofactor of 150, necessary for downstream processing. “FlowSom” clustering was performed using the cytometry analysis package, Catalyst (v1.14.1), with default parameters and all surface labels defined in the staining panel (excludes forward/side-scatter and viability) as features (Van Gassen et al, 2015, Cytometry Part A 87: 636-645). Dimension reduction to generate and visualize UMAPs were derived from the “runDR” and “plotDR” functions respectively, again using all fluorescent-based markers apart from viability.

### Presto Blue Viability

Primary human neutrophils and iPSC-derived neutrophils were resuspended in RPMI media supplemented with 10% FBS. 100,000 cells were plated into each well of a black clear-bottom 96-well plate. Separate plates were prepared for each timepoint (days 0, 1, 3 and 5). On day 0, cells were allowed to rest for at least 1 hour prior to addition of Presto Blue HS (Thermo Fisher #P50200). For each timepoint, Presto Blue HS was added and incubated for 30 minutes at 37° C. before reading fluorescence at 560/590 nm in a microplate reader (Synergy H1, Bio-Tek Instruments). Background fluorescence of media only wells was subtracted from each sample and the fold change was calculated compared to day 0, respective to each cell line.

### Chemotaxis

Chemotaxis was assessed using a microfluidic device as described previously (Yamahashi et al, 2015, Biomed. Microdevices 17: 100). In brief, polydimethylsiloxane (PDMS) devices were plasma treated and adhered to glass coverslips. Devices were coated with 10 μg/mL fibrinogen (Sigma) in PBS for 30 min at 37° C., 5% CO₂. The devices were blocked with 2% BSA-PBS for 30 min at 37° C., 5% CO₂, and then washed twice with mHBSS. Cells were stained with Calcein AM (Molecular Probes) in PBS for 10 min at room temperature followed by resuspension in modified HESS (+0.1% HSA+10 mM Hepes). Cells were seeded at 5×10⁶/mL to allow adherence for 30 min before addition of chemoattractant. Then, 3 uL of 1 μM fMLP (Sigma) chemoattractant was loaded into the input port of the microfluidic device. Cells were imaged every 30 seconds for 45-90 min on a Nikon Eclipse TE300 inverted fluorescent microscope with a 10× objective and an automated stage using MetaMorph software (Molecular Devices). Automated cell tracking analysis was done using JEX software (Warrick et al, 2016, PLos One 11: e0145081) to calculate chemotactic index and velocity.

Inflammatory Cytokine qRT-PCR

iPSC-differentiated neutrophils were stimulated with LPS to evaluate expression of inflammatory cytokines. A 6 well TC treated plate was pre-coated with 10 ug/ml fibronectin. Approximately 6 million cells resuspended in modified HBSS (mHBSS) were plated per well. Cells were allowed to rest for 30 min at 37° C. before stimulation. Final concentration of 200 ng/mL E. coli LPS (Sigma #L2755) was added to appropriate wells and the plate was incubated for 2 hours at 37° C. Floating cells were collected and spun down at 300×g. To collect RNA, 1 ml of Trizol (Invitrogen) was added to adherent cells and then combined with the pelleted floating cell sample. Samples were pipetted to mix and then stored at −80° C. until RNA extraction. RNA was isolated by Trizol extraction following manufacturer's instructions (Invitrogen #15596026). Collected RNA was stored at −80° C. until cDNA preparation. cDNA was synthesized using oligo-dT primers and the Superscript III First Strand Synthesis kit (Invitrogen #18080-051) following manufacturer's instructions. cDNA was used as the template for quantitative PCR (qPCR) using FastStart Essential Green DNA Master (Roche) and a LightCycler96 (Roche). Data were normalized to ef1a within each sample using the ΔΔCq method (Livak and Schmittgen, 2001, Methods 25: 402-408). Fold-change represents the change in cytokine expression over the unstimulated WT sample. All qPCR primers are listed in Table 3.

TABLE 3
qRT-PCR primers used in this study

| Gene | Sequence (5′ −> 3′) | Reference |
|---|---|---|
| EF1alpha | F-TGGTATTGGTACTGTTCCTG (SEQ ID NO. 1) | KiCqStart SYBR Green |
| | R-CTTCACTCAAAGCTTCATGG (SEQ ID NO. 2) | Primers |
| IL1beta | F-CTAAACAGATGAAGTGCTCC (SEQ ID NO. 3) | KiCqStart SYBR Green |
| | R-GGTCATTCTCCTGGAAGG (SEQ ID NO. 4) | Primers |
| IL6 | F-TCTCCACAAGCGCCTTCG (SEQ ID NO. 5) | PrimerDB |
| | R-CTCAGGGCTGAGATGCCG (SEQ ID NO. 6) | |
| IL8 | F-TGTAAACATGACTTCCAAGC (SEQ ID NO. 7) | KiCqStart SYBR Green |
| | R-AAAACTGCACCTTCACAC (SEQ ID NO. 8) | Primers |
| TNFa | F-GACAAGCCTGTAGCCCATGT (SEQ ID NO. 9) | Self-designed |
| | R-TCTCAGCTCCACGCCATT (SEQ ID NO. 10) | |

### IL-8 ELISA

iNeutrophil secretion of IL-8 was quantified using a human IL-8 ELISA following manufacturer's instructions (Bio-Techne #Dy208). A 12-well TC treated plate was coated with 10 ug/mL fibronectin. iNeutrophils were resuspended 2% FBS supplemented RPMI and 1.5 million cells were plated per well. Cells were allowed to rest for 30 min at 37° C. before stimulation. Cells were stimulated with 200 ng/mL E. coli LPS or 10 ug/mL Zymosan for 4 hours at 37° C. Wells were harvested and the media was spun down at 300×g to pellet the cells. Supernatants were collected, aliquoted and frozen at −80° C. until use.

### Phagocytosis

Phagocytosis was quantified using pHrodo™ Green E. coli BioParticles™ (Invitrogen #P35366) following manufacturer's instructions. Briefly, the E. coli BioParticles were opsonized with 30% pooled human serum (MP Biomedicals #MP092930149) for 30 minutes at 37° C. then washed 3 times in PBS. One million cells were resuspended in 80 uL of Media C and 20 uL of opsonized beads were added at 100:1 MOI. Cells and beads were incubated for 1 hour at 37° C., then stopped by addition of ice-cold PBS. While keeping tubes on ice, cells were stained with neutrophil lineage markers then fixed with 2% PFA before flow cytometry analysis on the Aurora Cytometer. Flow cytometry antibodies used can be found in Table 2.

### Intracellular ROS

Intracellular reactive oxygen species production of iPSC-differentiated neutrophils was quantified using the peroxynitrite indicator DHR123 (Invitrogen # D23806). A black wall clear-bottom 96-well plate was pre-coated with 10 ug/ml fibronectin. 100,000 cells in Phenol Red Free-RPMI containing 2% FBS and 5 ug/mL DHR123 reagent were plated into each well. PMA at a final concentration of 50 ng/mL was added to appropriate wells. Wells were plated in quadruplicate to account for technical error. Reads were taken every 15 minutes for 2 hours using the Victor3V microplate reader (PerkinElmer) to quantify intracellular peroxynitrite production (485/535 nm). Background signal of unstimulated cells was subtracted from the corresponding PMA stimulated cell line and plotted over time.

### NETosis Assay

NETosis or the release of extracellular DNA was quantified. A black walled clear-bottom 96-well microplate was pre-coated with 10 ug/mL Fibronectin. 200,000 cells in 100 uL Phenol Red Free RPMI+2% FBS were plated into each well. Cells were allowed to rest for 30 minutes at 37° C. Final concentration of 100 ng/mL PMA was added to appropriate wells and the plate was incubated for 4 hours at 37° C. Sytox Green (Invitrogen #S7020) was added at 375 nM final concentration. The microplate was incubated for 10 minutes and an endpoint reading was taken using a Victor3V microplate reader (PerkinElmer) to quantify extracellular DNA by fluorescence (500/528 nm). Background signal of Sytox Green unstimulated cells was subtracted from the corresponding PMA stimulated cell line. Fold change of fluorescence was calculated compared to WT.

### Production of Human Neutrophil-DC Hybrid Cells

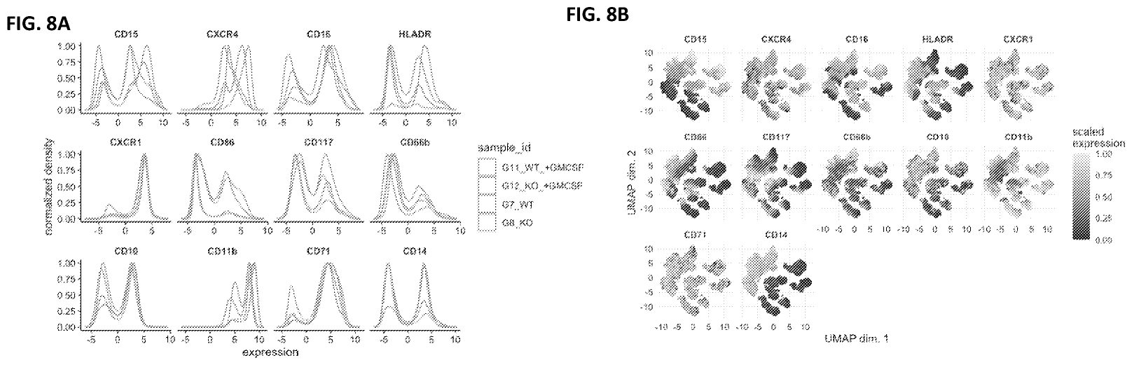

Neutrophils are produced as set forth above with the modification that, following culture of neutrophils in Media C for 6-8 days, cells were harvested and re-suspended in RPMI+10% FBS with or without 10 ng/mL GM-CSF. Alternatively, for a serum free option, cells can be re-suspended in Media C with added GM-CSF. Cells were cultured for an additional 2 days, then floating cells were harvested for further analysis. This protocol is illustrated in FIG. 8.

### Fungal Imaging

A. fumigatus (Af293) was grown on glucose minimal medium (GMM) plates at 37° C. in the dark to promote asexual conidiation. Aspergillus was plated at 1×10⁶ conidia/10 cm plate for 3-4 days. Conidia were harvested in 0.01% Tween water by scraping with an L-spreader and then passed through sterile Miracloth into a 50 ml conical tube. The spore suspension was centrifuged at 900×g for 10 min at room temperature and re-suspended in 50 ml 1×PBS. The spore suspension was then vacuum filtrated using a Buchner filter funnel with a glass disc containing 10-15 μm diameter pores. The filtered suspension was centrifuged at 900×g for 10 min and re-suspended in 1 ml 1×PBS. Conidia were counted using a hemacytometer and the concentration was adjusted to 1.5×10⁸ spores/ml. Conidial stocks were stored at 4° C. and used up to 1 month after harvesting.

Live imaging was conducted to visualize iPSC-derived neutrophil interactions with fungal hyphae. A. fumigatus (Af293) 2×10³ spores/well were plated in 100 uL GMM media in a black 24-well plate (Corning). The plate was incubated at 37° C. for 8 hours, or until germling stage. Spore germination was confirmed by microscopy prior to adding neutrophils. Primary neutrophils or iPSC-derived neutrophils were resuspended in RPMI+2% FBS at 6×10⁵ cells/mL. GMM media was removed from the wells and replaced with 100 uL of neutrophil suspension to yield a neutrophil to spore ratio of 150:1. Neutrophil-fungal interactions were imaged every 3 minutes on an inverted fluorescent microscope (Nikon Eclipse TE300) with a 20× objective and an automated stage (Ludl Electronic Products) with a Prime BSI Express camera (Teledyne Photometrics). Environmental controls were set to 37° C. with 5%C CO₂. Movies were compiled using ImageJ software.

### Bacterial Killing

P. aeruginosa (PAK strain) was diluted 1:50 from an overnight culture into LB media. The culture was shaken at 37° C. until the OD₆₀₀ was approximately 1.0. One mL of culture was spun down at 10,000×g, resuspended in 50% human serum-PBS, and shaken at 37° C. for 1 hour. Following incubation, the opsonized P. aeruginosa was washed 3× in PBS and the OD₆₀₀ was re-measured. 2×10⁶/mL iPSC-derived neutrophils were resuspended in Media C in an Eppendorf tube. P. aeruginosa was added 10:1 and co-incubated for 1 hour at 37° C. with shaking at 500 rpm. Bacteria only tube was included as a control. Eppendorf tubes were spun down 3 minutes at 500×g, washed in PBS, and then spun again to remove any extracellular bacteria. Triplicate CFUs were plated on LB agar at 10⁻³ and 10⁻⁴ dilutions to quantify remaining extracellular bacteria. Eppendorf tubes were spun down again for 3 minutes at 500×g and then resuspended in preheated 1.5 mL ddH₂O+1% Saponin+100 ug/mL DNase to lyse neutrophils. DNase is included to cleave extracellular DNA in the form of NETs that may kill viable bacteria. Samples were incubated at 37° C. for 10 minutes with shaking and intermittent vortexing. Eppendorf tubes were spun down at 8,000×g for 1 minute to pellet all cells. Colony forming units (CFUs) were plated on LB agar at 10⁻³ and 10⁻⁴ dilutions in triplicate to quantify viable P. aeruginosa. All CFU plates were incubated overnight at 37° C. Colonies were counted the next day and the average CFU/mL was determined for each sample.

### Fungal Killing Assay

C. auris yeast (1×10⁶ cells) and iPSC-derived neutrophils (1×10⁶ cells) were co-incubated for 6 hours in a black 96-well flat-bottom plate (Corning). Wells containing yeast alone and neutrophils alone were included as controls. After incubation, DNase I was added at a final concentration of 100 ug/mL to cleave any extracellular DNA. The plate was then incubated for 20 min at 37° C. with 5% CO₂. The total volume of each well was then transferred into a 96-well U-bottom plate to allow for efficient plate centrifugation and pelleting. As some neutrophils and yeast can adhere to the flat-bottom plate, the contents of both the U-bottom and flat-bottom plates were processed and ultimately combined for analysis. Following centrifugation of the U-bottom plate (1,200×g), 100 μl of a 100 μg/ml DNase I solution in ddH₂O was added to each well with pipette mixing and the plate was incubated 20 min to lyse neutrophils. The residual adherent cells in the flat-bottom plates were similarly treated. After incubation, the U-bottom plate was centrifuged to pellet C. auris, and supernatant containing lysed neutrophils was discarded. Then, the contents of the flat-bottom plate were removed and used to resuspend the pellets in the U-bottom plate, ensuring to keep the same well orientation between plates. Fresh DNase I solution was added to the flat-bottom plate, and both plates were incubated for 20 min. Then this process was repeated to ensure lysis of any remaining neutrophils. After this final incubation, supernatant was removed from the U-bottom plate, 90 μl of DPBS was added to the wells of the U-bottom plate with vigorous pipetting, and the contents were transferred to the corresponding wells in the flat-bottom plate. Then, a 1:10 dilution of PrestoBlue reagent was made in Phenol-red free RPMI+2% FBS and 110 μl of this solution was added to the wells of the U-bottom plate with vigorous pipette mixing. The contents were again transferred to corresponding wells in the flat-bottom plate containing yeast without viable neutrophils. The flat-bottom plate was incubated for 2 h at 37° C. with 5% CO2 before reading fluorescence at 560/590 nm in a microplate reader (Synergy H1, Bio-Tek Instruments). The percentage of viable yeast was quantified by calculating fluorescence signal from yeast incubated with neutrophils as a percentage of the same incubated without neutrophils. The background fluorescence levels of neutrophil-only controls were subtracted from the values of wells containing neutrophils and yeast.

For A. fumigatus killing, 2.5×10⁵ spores/well were plated in 100 uL GMM media in a black 96-well plate (Corning). The plate was incubated at 28° C. for 12 hours to allow for germination. Spore germination was confirmed by microscopy prior to adding neutrophils. Primary neutrophils or iPSC-derived neutrophils were resuspended in RPMI+2% FBS at 5×10⁶ cells/mL. GMM media was removed from the 96 well plate and replaced with 100 uL of neutrophil suspension. Wells containing yeast alone and neutrophils alone were included as controls. The plate was incubated at 37° C. for 4 hours. 100 uL of 100 μg/ml DNase I solution in ddH₂O was added and incubated for 20 minutes at RT to lyse the neutrophils and remove NETs. Fresh DNase solution was added and incubated for 20 minutes, then repeated again with a 10-minute incubation. Wells were washed 3× with ddH₂O to remove dead cells stuck to hyphae, being careful not to disrupt hyphae stuck to the plate. PrestoBlue reagent was diluted 1:10 in RPMI+2% FBS and added 100 ul to each well. The plate was incubated at 37° C. and fluorescence read at 560/590 nm in a plate reader after 2 h. The percentage of viable yeast was quantified by calculating fluorescence signal from yeast incubated with neutrophils divided by the fluorescent signal of yeast alone.

### Results

Generation of PTP1B Null iPSC-Derived Neutrophils.

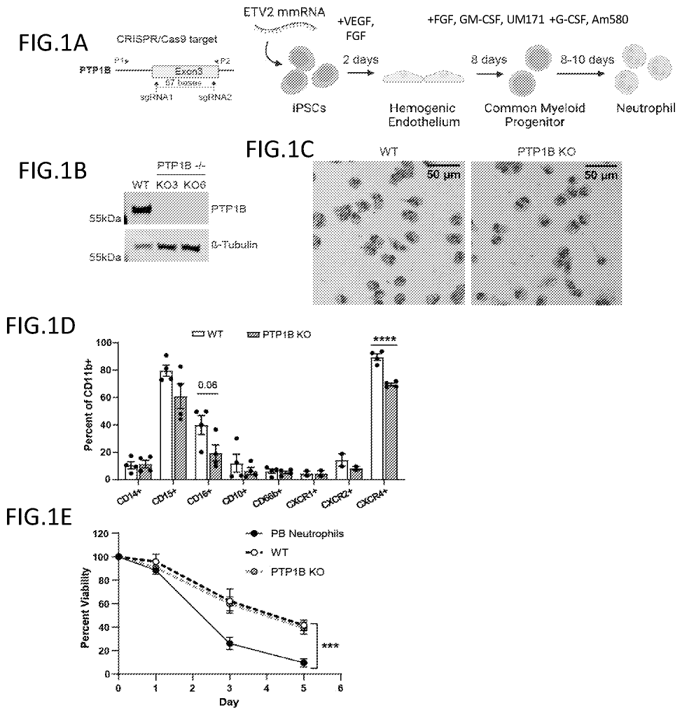

Human iPSC-derived neutrophils were generated in serum-and feeder-free conditions following published protocols (FIG. 1A) (Brok-Volchanskaya et al., 2019, Stem Cell Reports 13:1099-110; Majumder et al., 2020, STAR Protoc. 1(2)). To increase activation of intracellular signaling pathways in iPSC-derived neutrophils, protein tyrosine phosphatase 1B (PTP1B) genes were deleted using CRISPR/Cas9 mediated gene mutation at the iPSC-stage (FIG. 1A). Loss of PTP1B protein expression was confirmed after neutrophil differentiation by western blot (FIG. 1B). Morphological characteristics of differentiated neutrophils was examined by cytospin and confirmed the presence of hyper-segmented nuclei (FIG. 1C). Further validation of neutrophil differentiation was completed by staining for neutrophil surface receptors. iPSC-derived neutrophils exhibit low levels of CD10 and CD66b, but exhibit higher expression of CD15 and CD16 (FIG. 1D) (Lachmann et al., 2015, Stem Cell Reports. 4:282; Brok-Volchanskaya, 2019, Id.). While, deletion of PTP1B−/− resulted in lower numbers of fully mature CD16+ neutrophils, the majority of cells still expressed the neutrophil marker CD15 (FIG. 1D). PTP1B has been shown to modify murine myelopoiesis by negatively regulating monocyte differentiation (Heinonen et al., 2006, Proc Natl Acad Sci USA. 103:2776-81). No significant increase in CD14+ monocyte differentiation was observed upon deletion of PTP1B (FIG. 1D).

To validate the longevity of iPSC-derived neutrophils over primary human peripheral blood (PB) neutrophils, lifespan of these cells were evaluated in culture. PB neutrophils have a short lifespan ex vivo exhibiting less than 30% viability at 3 days, whereas iPSC-derived neutrophils still exhibit 50% viability after 5 days, with no difference between WT and PTP1B null cell lines (FIG. 1E). Data indicates that deletion of PTP1B still allows for neutrophil differentiation but decreases the maturation of these cells.

### Deletion of PTP1B Increases IL-8 Inflammatory Cytokine Production

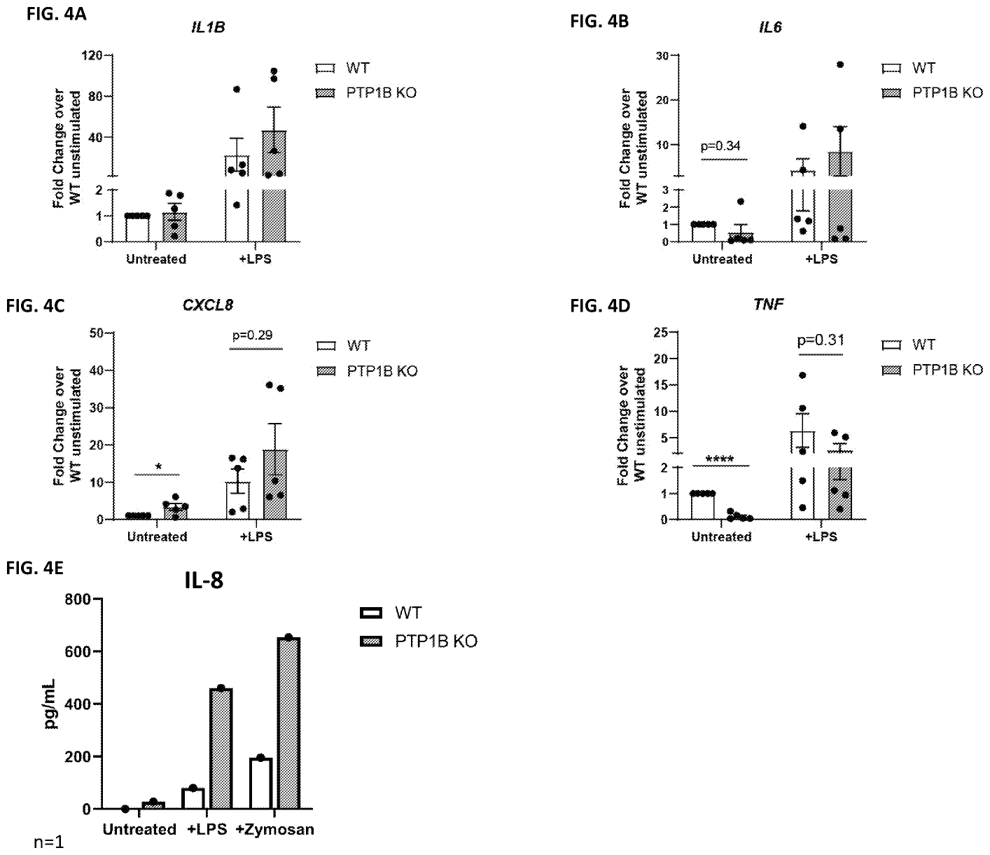

Upon migrating to sites of infection, neutrophils can produce inflammatory cytokines to further recruit and promote the innate and adaptive immune response. PTP1B-null iNeutrophils displayed differences in inflammatory cytokines at basal levels, quantified by qPCR. While IL6 and TNF were significantly decreased basally, CXCL8 was significantly increased (FIG. 4A-4D). These data indicated a role for PTPT1B in regulating cytokine gene expression. In response to LPS stimulation, PTP1B-null iPSC-derived neutrophils produced similar levels of inflammatory gene transcripts as WT cells (FIG. 4A-4D). While not statistically significant, CXCL8 was increased over WT cells after LPS stimulation in the majority of replicates (FIG. 4C). To confirm translation to protein, secretion of IL-8 was quantified by ELISA and confirmed that PTP1B-null neutrophils produced more IL-8 when stimulated with LPS or zymosan (FIG. 4E). Increased IL-8 production promoted neutrophil activation and recruitment, as IL-8 is a strong neutrophil chemoattractant. Elevated production of IL-6 and TNFa have previously been found in LPS stimulated PTP1B-null murine BM-DCs and macrophages (Yue et al., 2016, Am J Pathol. 186:1234-44; Xu et al., 2008, Mol Immunol. 45:3545-52). These data indicated that PTP1B can differentially regulate production of these inflammatory cytokines in human neutrophils.

### Deletion of PTP1B Promotes Intracellular Signaling and Neutrophil Motility

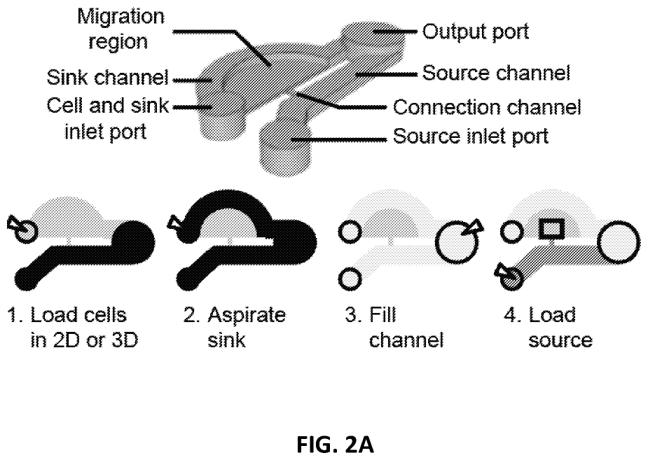

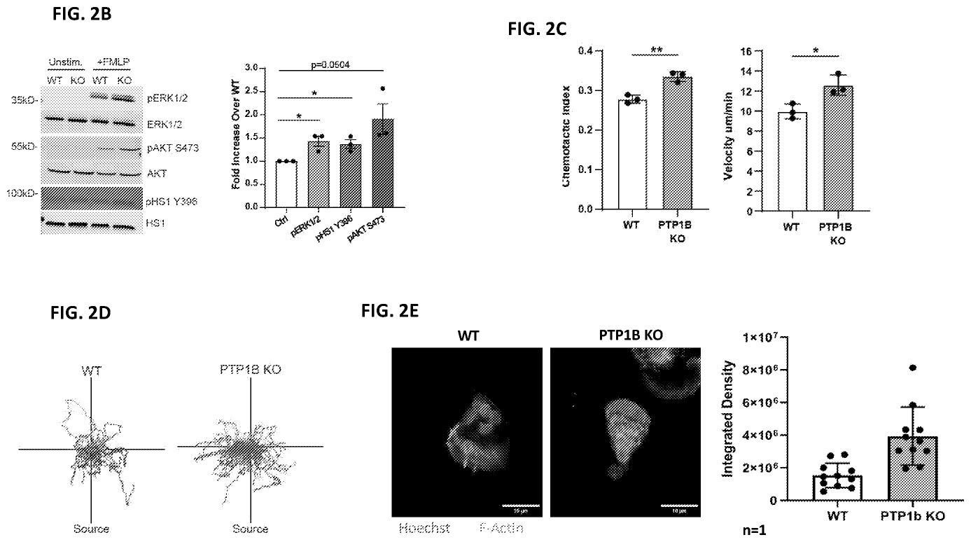

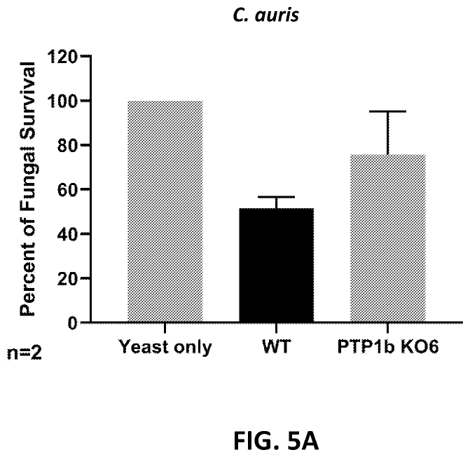

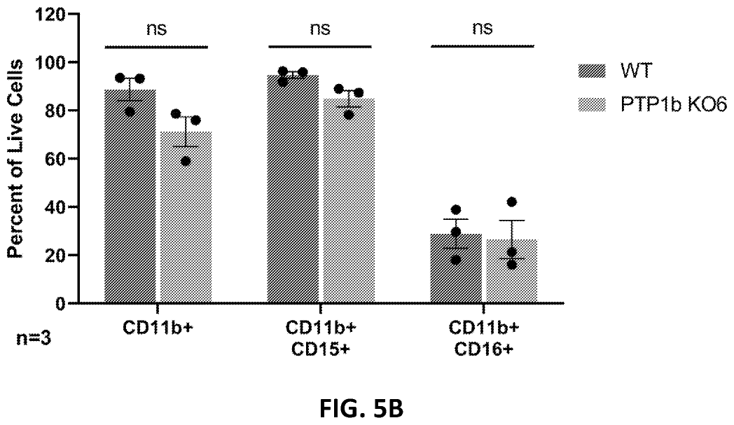

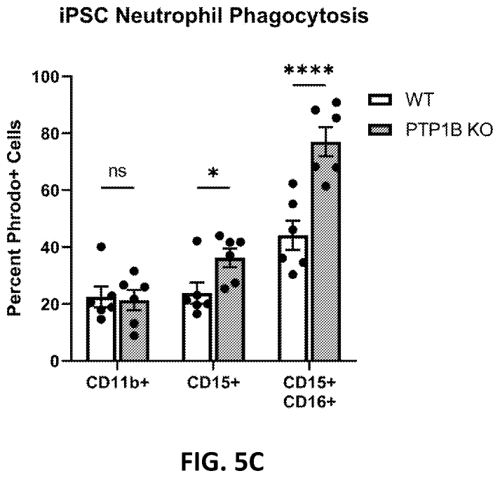

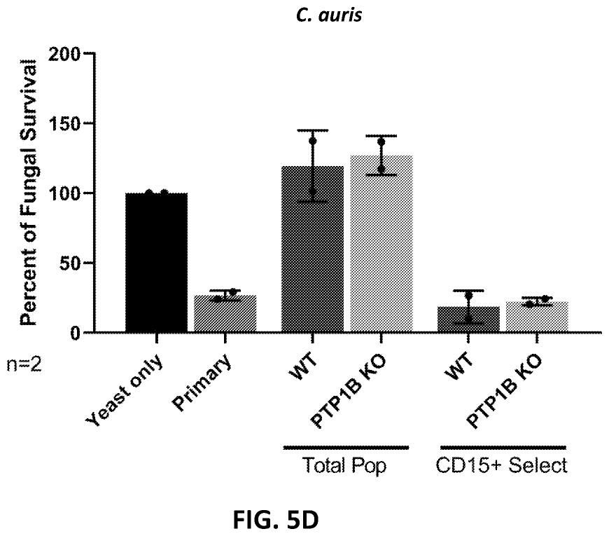

Upon validating differentiation of PTP1P-null neutrophils, deletion of the PTP1B phosphatase was functionally confirmed in these iPSC-derived neutrophils. Phospho-signaling was evaluated upon stimulation with the bacterial formylated peptide FMLP and determined that PTP1B−/− neutrophils show greater phosphorylated ERK1/2, HS1, and AKT compared to WT (FIG. 2A). Thus, deletion of PTP1B produces increases intracellular signaling within iPSC-derived neutrophils. Enhanced phospho-signaling, including the actin regulatory protein HS1 may promote neutrophil chemotaxis (Cavnar et al., 2012, J Biol Chem. 287:25466-77). Using a microfluidic device known in the art (Berthier et al., 2010, Integr Biol (Camb). 2:630-8), neutrophil migration was live imaged in response to the chemoattractant FMLP. PTP1B-null neutrophils displayed enhanced motility with higher chemotactic index and velocity compared to WT (FIG. 2B). Representative cell tracks show increased directed migration of PTP1B-null iNeutrophils (FIG. 2C). As PTP1B-KO cells have increased p-HS1 and enhanced migration after stimulation, it was determined whether these cells show changes in actin polarization. During migration, actin is polymerized at the lead edge to drive pseudopod formation (Hind et al., 2016, Dev Cell. 8:161-9)). F-actin was imaged after stimulation with FMLP. PTP1B-KO cells displayed increased polarized actin at the leading edge compared to WT cells (FIG. 2D). Thus, deletion of the PTP1B phosphatase increases intracellular signaling leading to increased actin polarization and enhanced neutrophil motility. Neutrophils exhibit many antimicrobial effector functions, including the ability to phagocytose microbes. Phagocytosis is mediated by actin contraction to form the phagosome (May & Machesky, 2001, J. Cell Sci. 114: 1061-1077) and thus may be enhanced in PTP1B-KO iNeutrophils. Phagocytosis of E. coli coated beads was quantified by flow cytometry and found a significant increase in phagocytosis by CD15+ PTP1B-null neutrophils (FIG. 5C). This effect was heightened when gating on CD16+ mature neutrophils with close to 80% of PTP1B-null neutrophils phagocytosing E. coli coated beads, whereas approximately 40% of WT CD15+ neutrophils had phagocytosed after 1 hour. Deletion of PTP1B has previously been shown to promote neutrophil phagocytosis in murine models (Yue et al., 2019, PLoS One 14: e0222753)), but it was unknown if human neutrophils responded similarly. The results set forth herein showed PTP1B negatively regulated Fc-receptor mediated phagocytosis in human neutrophils.

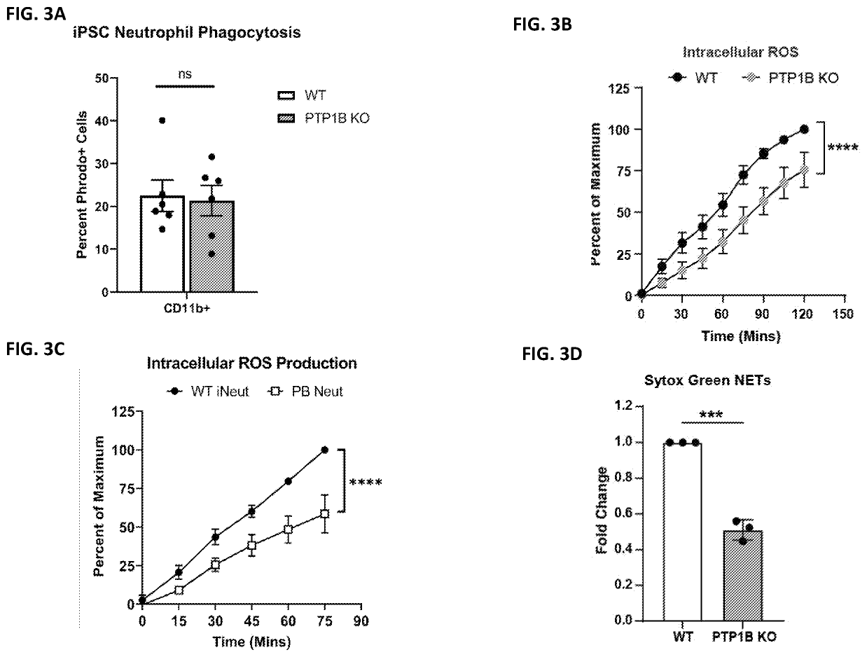

Following phagocytosis, neutrophils can kill pathogens by production of intracellular reactive oxygen species (ROS). While PTP1B-null neutrophils produced lower levels of ROS than WT (FIG. 3B), iPSC-derived neutrophils are more potent producers of ROS than primary neutrophils (FIG. 3C). PTP1B-null neutrophils were still capable of producing ROS at high levels upon PMA stimulation and likely produce levels similar to that of primary human neutrophils (FIG. 3B and FIG. 3C).

Another mechanism for neutrophil antimicrobial killing is the release of neutrophil extracellular traps (NETS). iNeutrophils were stimulated with PMA and it was found that PTP1B-null neutrophils NETose at significantly lower levels (FIG. 2E). Abundant induction of NETs can lead to tissue damage and are associated with autoimmune diseases (Branzk et al., 2014, Nat Immunol. 15:1017-25; Castanheira et al., 2019, Blood 133:2178-85). Thus, lower NET production in PTP1B-null neutrophils can be expected to reduce neutrophil-mediated damage in patient tissues. PTP1B-null neutrophils produce sufficient ROS and have enhanced phagocytosis which could promote pathogen clearance in patients.

FIG. 5A-5D shows that selection for mature CD15+ neutrophils increased PTP1B-null neutrophil function. Co-culture of iNeutrophils with the fungal pathogen C. auris shows that both WT and PTP1B KO neutrophils can kill fungus. Wild type (WT) neutrophils had slightly improved killing (FIG. 5A), due to reduced CD15± mature neutrophil differentiation in the PTP1B KO (FIG. 1D). Thus, CD15+ neutrophils can be positively selected to evaluate the effect of PTP1B deletion on neutrophil function in mature neutrophils only. PTP1b KO neutrophils after CD15+ bead selection for mature neutrophils had similar neutrophil marker expression compared to wild-type neutrophils (FIG. 5B). PTP1B KO phagocytosis was greatly enhanced when gating on CD15+ or CD16+ neutrophils and was significantly greater than wild-type neutrophils (FIG. 5C). Additionally, PTP1b KO neutrophils after CD15+ bead selection for mature neutrophils had fungal killing comparable to wild-type neutrophils and primary human neutrophils.

iNeutrophil Differentiation With GM-CSF Treatment

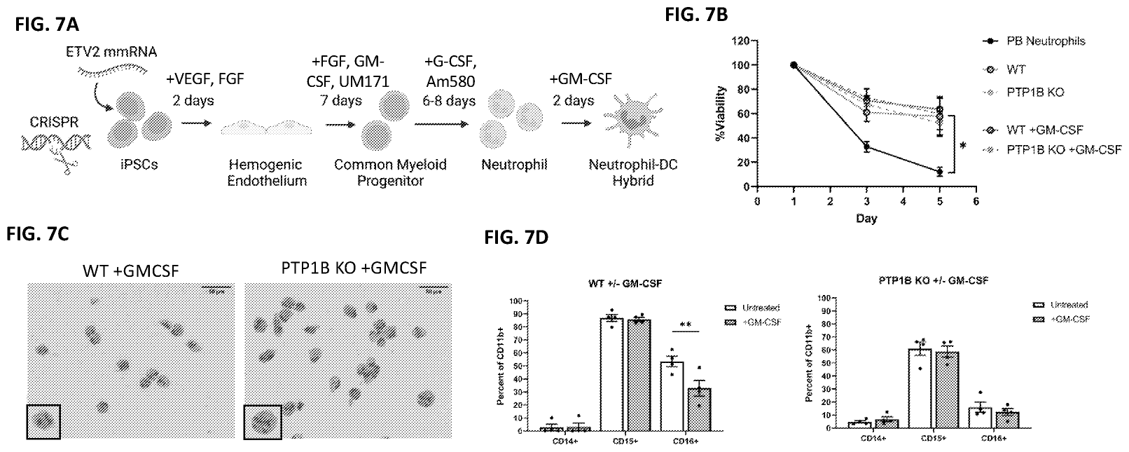

GM-CSF treatment of primary human neutrophils for up to 48 hours increases expression of antigen presenting markers (HLA-DR, CD80 and CD86) (Chakravarti et al., 2009, Lab Invest. 89:1084-99; Fanger et al., 1997, Blood 89:4128-35; Matsushima et al., 2013, Blood 121:1677-89). To generate neutrophil-DC hybrids from iPSCs, the standard differentiation protocol (FIG. 1A) was utilized, with an additional two days supplemented with 10 ng/mL GM-CSF. iPSC-derived neutrophils are beneficial for therapeutic use over primary human neutrophils due to their extended lifespan (FIG. 7B), as only a small proportion of primary neutrophils can survive the 48-hour GM-CSF treatment (Chakravarti et al., 2009, Id.). While iNeutrophils have greater longevity over primary human neutrophils, treatment with GM-CSF had no effect on viability after 5 days (FIG. 7B). Cytospin stains indicated that iNeutrophils treated with GM-CSF still differentiated with the typical hypersegmented nuclei of primary human neutrophils (FIG. 7C). Neutrophil differentiation was further evaluated by quantifying lineage markers by flow cytometry. GM-CSF treatment has been shown to promote shedding of the CD16 receptor (Moulding et al., 1999, J Leukoc Biol. 65:875-82)). While wild-type (WT) cells exhibited a slight decrease in expression of CD16 with GM-CSF treatment, PTP1B−/− neutrophils were generated in similar numbers with or without GM-CSF treatment (FIG. 7D).

### Characterization of Human Neutrophil-DC Hybrid Cells

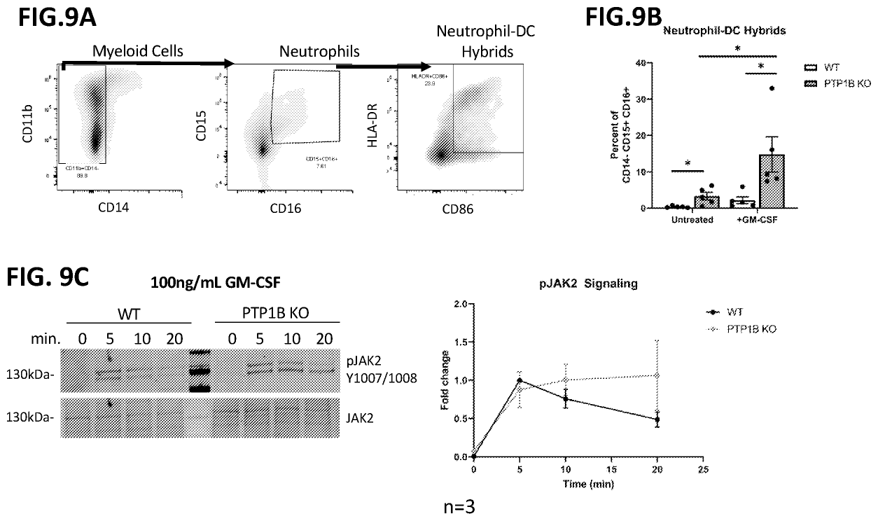

Expression of dendritic cell markers CD86 and HLADR were evaluated in cells produced by incubation of iPSC-derived neutrophils with GM-CSF. Wild type neutrophils displayed a mild increase in CD86+HLADR+ with ˜1% of neutrophils expressing both markers, while PTP1B knockout resulted in ˜15% of neutrophils expressing both markers. These results are shown in FIG. 9B.

### Antifungal Killing Using Human Neutrophil-DC Hybrid Cells

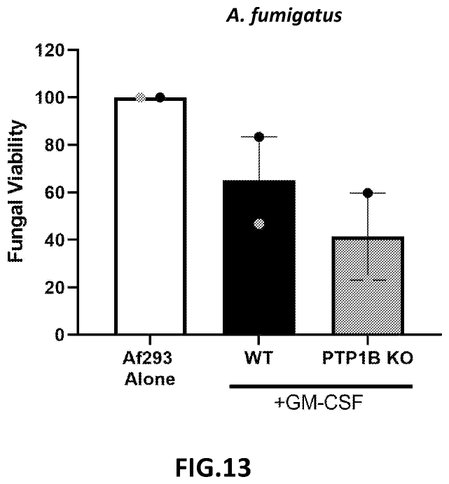

Capacity of human neutrophil-DC hybrid cells to kill fungal pathogens was evaluated by co-incubation with A. fumigatus. As shown in FIG. 13, PTP1B KO neutrophil-DC hybrids had improved fungal killing compared to wild-type neutrophil-DC hybrids treated with GM-CSF.

These results indicated that neutrophil-DC hybrid cells produced from cells not expressing or having reduced expression of PTP1B phosphatase exhibited a more effective fungal killing phenotype neutrophil-DC hybrids having a wild-type expression phenotype of PTP1B phosphatase. These cells were shown to have improved functional capacity in enhanced fungal killing, which could provide improved capacity against fungal infections in neutropenic patients. Furthermore, because neutrophil-DC hybrids have been identified after radiotherapy-radiodynamic therapy in mouse tumor models and were shown to decrease tumor burdens (Guo et al., 2021, ACS Nano 15: 17515-17527), the iPSC-derived PTP1B KO neutrophil-DC hybrid cells can also have cellular immunotherapy applications.

PTP1B KO Neutrophils Show Increased Activation and Swarming in Response to A. fumigatus

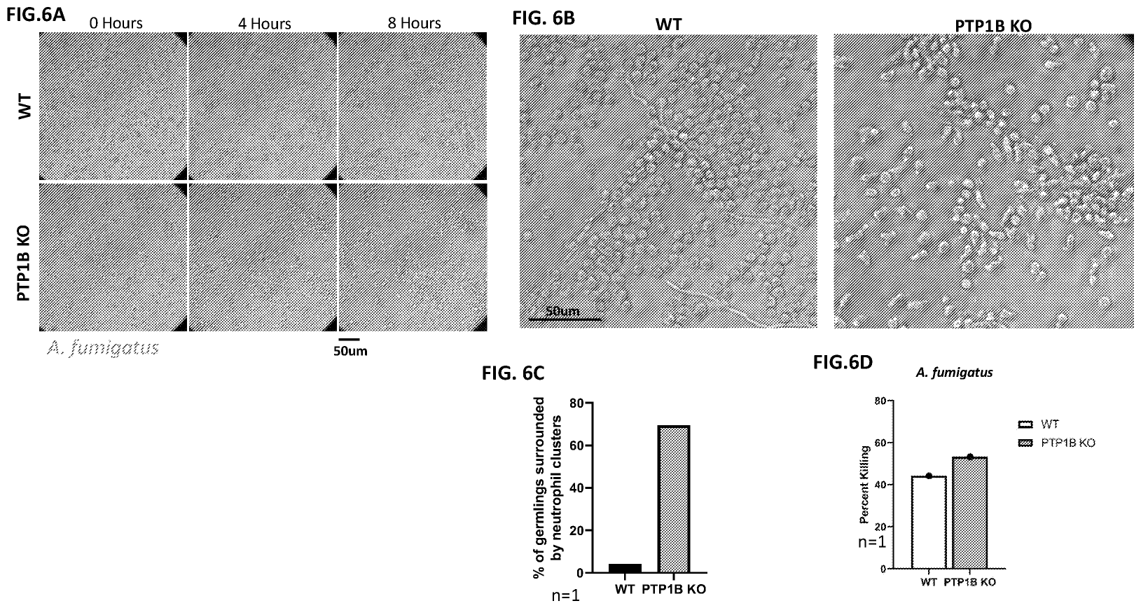

To determine the overall impact on iNeutrophil response to live fungal pathogen, A. fumigatus was co-cultured with iNeutrophils at the germling stage and then live imaged over the course of 8 hours (FIG. 6A). A stark difference in the morphology of WT vs PTP1B-null iNeutrophils was observed in the presence of A. fumigatus. PTP1B-null iNeutrophils displayed an elongated cell shape indicative of cell activation and attachment to the fibronectin substrate (FIG. 6B). In contrast, WT cells remained inactive with a rounded morphology and did not appear to attach to the plate (FIG. 6B). It was also found that PTP1B-null iNeutrophils were highly migratory and began to attach to and surround the hyphae, whereas very few WT iNeutrophils physically interacted with the hyphae. Increased activation by A. fumigatus in the PTP1B-null iNeutrophils may be due to increased expression of pathogen recognition receptors (PRR) such as dectin-1 or other TLRs. Neutrophil clustering around A. fumigatus germlings was quantified and it was found that approximately 70% of germlings were surrounded by PTP1B-null iNeutrophils, whereas less than 5% were surround by WT iNeutrophils (FIG. 6C). These clusters likely indicated neutrophil swarming behavior mediated by release of LTB4 (Kienle & Lammermann, 2016, Immunol Rev. 273:76-93.). Increased swarming by PTP1B-null iNeutrophils could indicate increased production of LTB4 or expression of its receptors BLT1 and BLT2. Fungal killing was evaluated by the iPSC-derived neutrophils. After 4-hour coculture, WT iNeutrophils killed 45% of A. fumigatus, whereas PTP1B-null cells killed at a higher rate with 55% killing (FIG. 5D). Increased cell activation and swarming by PTP1B-null iNeutrophils likely contributed to increased fungal killing.

GM-CSF Treatment of iNeutrophils Induces Expression of Antigen Presenting Markers.

As differentiation of neutrophil-DC hybrids may not be homogenous, these cells were stained with a larger flow cytometry panel of neutrophil markers and antigen presenting markers (HLA-DR, CD86). Expression of CD86 and HLADR were increased in PTP1B KO cells and were elevated further with GM-CSF treatment, compared to WT (FIG. 8A). Additionally, CXCR4 expression was decreased upon GM-CSF treatment for both cell lines (FIG. 8A). CXCR4 is typically considered to be a marker of aged neutrophils. It is decreased upon neutrophil maturation in the bone marrow, then increases with age to act as a homing factor for neutrophil recruitment back to the bone marrow (DeFillippo & Rankin, 2018, Eur J Clin Invest 48 Suppl 2:e12949). However, the implications of CXCR4 expression on iPSC-derived neutrophils is unclear as these cells did not arise from the bone marrow niche. Cell populations were further analyzed by clustering individual cells based upon surface receptor expression. A population of CD15+ CD16+ CD14− neutrophils expressing antigen presenting markers CD86 and HLADR (FIG. 8B) was identified. Lack of CD14 expression indicates that these are not monocytes, which typically expressCD86 and HLADR. Thus, this combination of surface markers was utilized to identify a population of neutrophil-DC hybrids.

### PTP1B Knockout Increases Differentiation of Neutrophil-DC Hybrids Via JAK2 Signaling

To quantify the proportion of neutrophil-DC hybrids generated in culture CD14− CD15+CD16+HLADR+CD86+ cells (FIG. 9A) were gated on WT cells differentiated less than 1% of neutrophils into neutrophil-DC hybrids. This was slightly increased with GM-CSF stimulation (FIG. 9B). In contrast, a significant increase in neutrophil-DC hybrids generated even from untreated PTP1B−/− iPSCs was observed, with 3.5% of cells expressing antigen presenting markers (FIG. 9B). Upon GM-CSF treatment, 15% of all PTP1B−/− neutrophils differentiated into neutrophil-DC hybrids, a significant increase over 2% of WT cells (FIG. 9B). Thus, deletion of PTP1B increases expression of HLA-DR MHC class II and CD86 with GM-CSF treatment, allowing for more efficient neutrophil-DC hybrid differentiation.

To understand the mechanism that promotes expression of antigen presenting markers HLA-DR and CD86 on PTP1B−/− neutrophils, intracellular signaling following GM-CSF stimulation was evaluated. PTP1B has been shown to dephosphorylate JAK2 on receptors such as CSF1R (Heinonen et al., 2006, Proc Natl Acad Sci U S A. 103:2776-81; Villamar-Cruz et al., 2021, Biosci Rep. 41(11): BSR20211994). GM-CSF signaling requires JAK2 kinase activity and thus may be targeted by PTP1B dephosphorylation to limit downstream signaling. Upon stimulation of iPSC-derived neutrophils with 100 ng/mL GM-CSF, no significant increase in pJAK2 signaling in PTP1B KO neutrophils was observed after 5 minutes (FIG. 9C). However, PTP1B KO neutrophils showed prolonged phosphorylated JAK2 at 10 and 20 minutes post-stimulation, compared to WT (FIG. 9C). Thus, increased differentiation of PTP1B KO neutrophils into DC hybrids may be due to enhanced GM-CSF receptor signaling.

iPSC-Derived Neutrophil-DC Hybrids Have Enhanced Phagocytosis

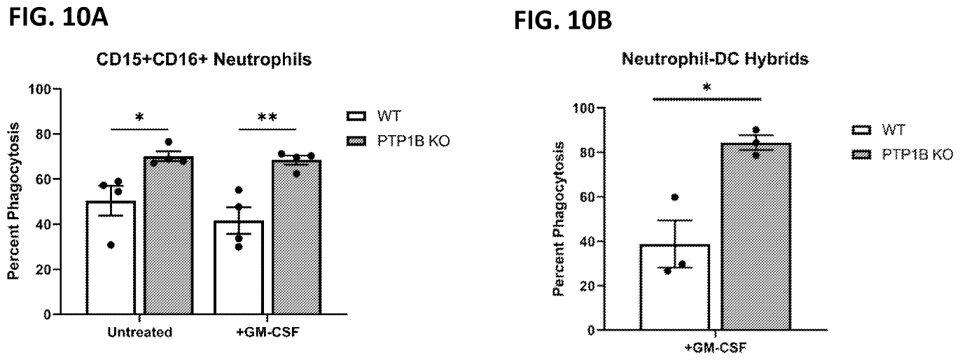

Neutrophil-DC hybrids have previously been shown to exhibit enhanced phagocytosis over untreated neutrophils (Fites et al., 2018, PLoS Pathog. 14:e1007073). As shown in FIG. 5C, deletion of PTP1B increases CD15+ iNeutrophil phagocytosis over WT cells. As PTP1B-null neutrophils have increased signaling upon stimulation with GM-CSF, whether GM-CSF treatment would improve iNeutrophil phagocytosis even further was determined. Treatment with GM-CSF did not affect iNeutrophil phagocytosis with similar levels to untreated cells for both WT and PTP1B-No neutrophils (FIG. 10A). However, upon evaluating phagocytosis by the neutrophil-DC hybrids, a significant enhancement was seen by the PTP1B-null cells with over 80% of cells positive for acidified beads (FIG. 10B).

### PTP1B-Null Neutrophils Have Increased Expression of Granule Markers

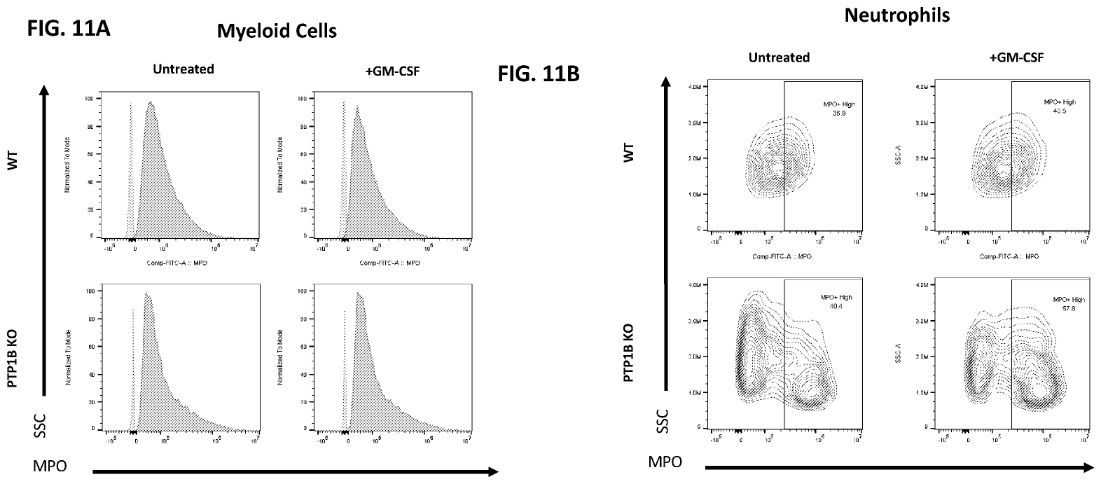

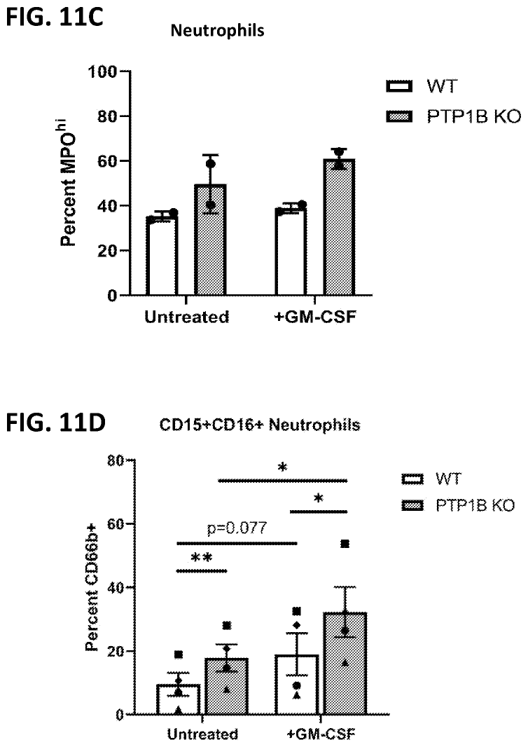

Neutrophils contain secretory granules that can be released upon stimulation to kill extracellular pathogens. To determine if PTP1B-null neutrophils or GM-CSF stimulation would affect neutrophil granules, granule markers on iNeutrophils were evaluated. Azurophilic (primary) granules contain myeloperoxidase (MPO) and elastase antimicrobial proteins (Metzler et al., 2011, Blood 117:953-9). Intracellular staining by flow cytometry indicated that close to 100% of all iNeutrophils contain MPO (FIG. 11A). However, upon closer analysis, a population of cells with high expression of MPO (MPOhi) was identified (FIG. 11B). PTP1B-null neutrophils showed an increased proportion of MPOhi neutrophils compared to WT cells (FIG. 11C). Upon stimulation with GM-CSF, the percent of MPOhi PTP1B-null iNeutrophils slightly increased (FIG. 11C). Furthermore, when expression of CD66b was evaluated, an indicator of specific (secondary) granules (Lacy, 2006, Allergy Asthma Clin Immunol. 2: 98-108), a similar pattern was found. CD66b expression among CD11b+ myeloid cells was comparable between WT and PTP1B-null iNeutrophils (FIG. 1D). However, a population of CD66b+ cells that were significantly increased in PTP1B-KO CD15+ iNeutrophils (FIG. 11D) was identified. A further increase in the proportion of CD66b+ cells upon GM-CSF treatment of PTP1B-null iNeutrophils (FIG. 11D) was seen. Thus, PTP1B can negatively regulate granule development or expression of granule proteins. Elevated MPO and CD66b expression in PTP1B-deleted iNeutrophils could correlate to increased degranulation upon stimulation. Furthermore, CD66b is often characterized as a neutrophil activation marker and correlates to the increased cell activation of PTP1B-null neutrophils co-incubated with A. fumigatus (FIG. 6A-6D). Increased neutrophil activation and release of MPO may improve PTP1B-null neutrophil killing of extracellular pathogens.

GM-CSF Treatment Improves Neutrophil Control of A. fumigatus

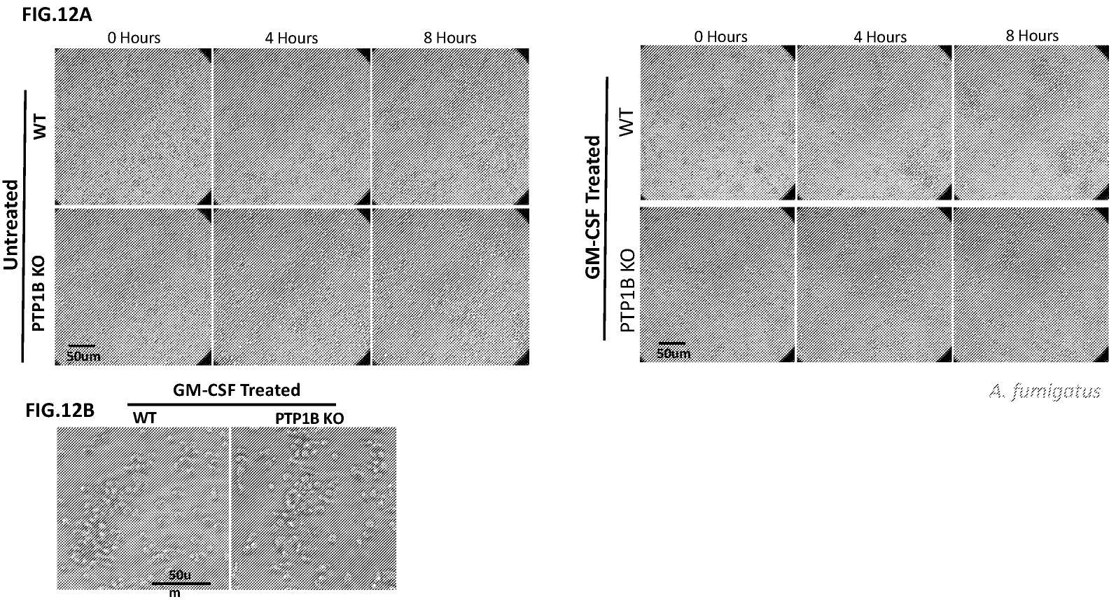

To determine the impact of GM-CSF treatment on iNeutrophil control of the fungal pathogen A. fumigatus, a co-culture of these cells was live imaged over 8 hours. Representative images over the time course show decreased fungal burden with GM-CSF treated iNeutrophils compared to untreated iNeutrophils (FIG. 12A). It was previously shown that untreated WT iNeutrophils remain round and inactivated in the presence of A. fumigatus (FIG. 6B). Upon GM-CSF treatment, an increased activation of WT cells indicated by an elongated cell shape and increased cell migration (FIG. 12B) was seen. Thus, GM-CSF treatment can be expected to prime wild-type iNeutrophils to enhance their recognition and response to A. fumigatus.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description can be made without departing from the spirit or scope of the present invention, as defined in the following claims.

1. A method of producing modified, mature neutrophils from pluripotent stem cells, the method comprising:
(a) transiently introducing exogenous ETV2 in human pluripotent stem cells having inhibited expression of protein tyrosine phosphatase 1B (PTP1b) and culturing the ETV2-pluripotent stem cells in serum-free culture medium comprising vascular endothelial growth factor-165 (VEGF-165) and fibroblast growth factor 2 (FGF2), to produce a population of ETV2-hemogenic endothelial cells (ETV2-HECs);
(b) culturing the ETV2-HECs in serum-free and xeno-free culture medium comprising granulocyte-macrophage colony-stimulating factor (GM-CSF) and FGF2 for a sufficient time to produce myeloid progenitors;
(c) culturing the myeloid progenitors in serum-free and xeno-free culture medium comprising granulocyte-colony stimulating factor (G-CSF) and retinoic acid receptor agonist for a time sufficient to differentiate the myeloid progenitors into a population of modified mature neutrophils; and
(d) selecting mature neutrophils from the population of modified mature neutrophils.

2. The method of claim 1, wherein the serum-free and xeno-free culture medium in (b) further comprises UM171.

3. The method of claim 1, wherein the expression of PTP1b is inhibited in the human pluripotent stem cells by gene mutation, RNA-mediated inhibition, RNA editing, DNA gene editing or base editing.

4. The method of claim 1, wherein the gene editing method comprises sequence modification using a nuclease selected from a meganuclease, ZFNs, TALENs, and Cas enzyme.

5. The method of claim 4, wherein the nuclease is a Cas9 enzyme.

6. The method of claim 1, wherein the retinoic acid receptor agonist is AM580.

7. The method of claim 1, wherein the method comprises one or more of the following:
step (a) comprises culturing the ETV2-pluripotent stem cells for about 1 to 2 days;
step (b) comprises culturing the ETV2-HECs for about 7 to 12 days; and
steps (c) and (d) comprising culturing the myeloid progenitor cells for about 6 to 8 days.

8. The method of claim 1, wherein the pluripotent stem cells are induced pluripotent stem cells.

9. The method of claim 1, wherein the mature neutrophils express CD15 and/or CD16.

10. The method of claim 1, wherein selecting comprises a cell separation, cell sorting, or enrichment method.

11. A population of modified mature neutrophils produced by the method of claim 1.

12. A method of treating cancer comprising administering to a patient in need thereof a therapeutically effective amount of the modified mature neutrophils of claim 11.

13. A method of treating bacterial infection comprising administering to a patient in need thereof a therapeutically effective amount of the modified mature neutrophils of claim 11.

14. The method of claim 13, wherein the bacterial infection is a systemic infection.

15. A pluripotent stem cell having inhibited expression of protein tyrosine phosphatase 1B (PTP1b).

16. The pluripotent stem cell of claim 15, wherein the expression of PTP1b is inhibited in the human pluripotent stem cells by gene mutation, RNA-mediated inhibition, RNA editing, DNA gene editing or base editing.

17. The pluripotent stem cell of claim 15, wherein the gene editing method comprises sequence modification using a nuclease selected from a meganuclease, ZFNs, TALENs, and Cas enzyme.

18. The pluripotent stem cell of claim 17, wherein the nuclease is a Cas9 enzyme.

19. A method for producing human neutrophil-dendritic (DC) cell hybrids in vitro comprising the steps of:
(a) transiently introducing exogenous ETV2 in both wild-type human pluripotent stem cells and cells having inhibited expression of protein tyrosine phosphatase 1B (PTP1b) and culturing the ETV2- pluripotent stem cells in serum-free culture medium comprising vascular endothelial growth factor-165 (VEGF-165) and fibroblast growth factor 2 (FGF2), to produce a population of ETV2-hemogenic endothelial cells (ETV2-HECs);
(b) culturing the ETV2-HECs in serum-free and xeno-free culture medium comprising granulocyte-macrophage colony-stimulating factor (GM-CSF) and FGF2 for a sufficient time to produce myeloid progenitors;
(c) culturing the myeloid progenitors in serum-free and xeno-free culture medium comprising granulocyte-colony stimulating factor (G-CSF) and retinoic acid receptor agonist for a time sufficient to differentiate the myeloid progenitors into a population of neutrophils;
(d) harvesting the neutrophil population and resuspending the cells in serum-supplemented or serum-free culture media with comprising GM-CSF for an additional two days to produce human neutrophil-DC hybrid cells; and
(e) selecting human neutrophil-DC hybrid cells from the culture.

20. The method of claim 1, wherein the culture medium in step (b) further comprises UM171.

21. The method of claim 19, wherein the method comprises one or more of the following:
step (a) comprises culturing the ETV2-pluripotent stem cells for about 1 to 2 days;
step (b) comprises culturing the ETV2-HECs for about 7 to 12 days;
step (c) comprises culturing the myeloid progenitor cells for about 6 to 8 days; and
step (d) comprises culturing the neutrophil population for about 2 days.

22. The method of claim 19, wherein the pluripotent stem cells are induced pluripotent stem cells.

23. The method of claim 19, wherein the human neutrophil-DC hybrid cells express CD86 and HLADR.

24. A population of human neutrophil-DC hybrid cells produced by the method of claim 19.


SEQUENCE LISTING (15 sequences)

SEQ ID NO: 1 - DNA, 20 bp, synthetic construct
  source 1..20: mol_type = other DNA
tggtattggtactgttcctg

SEQ ID NO: 2 - DNA, 20 bp, synthetic construct
  source 1..20: mol_type = other DNA
cttcactcaaagcttcatgg

SEQ ID NO: 3 - DNA, 20 bp, synthetic construct
  source 1..20: mol_type = other DNA
ctaaacagatgaagtgctcc

SEQ ID NO: 4 - DNA, 18 bp, synthetic construct
  source 1..18: mol_type = other DNA
ggtcattctcctggaagg

SEQ ID NO: 5 - DNA, 18 bp, synthetic construct
  source 1..18: mol_type = other DNA
tctccacaagcgccttcg

SEQ ID NO: 6 - DNA, 18 bp, synthetic construct
  source 1..18: mol_type = other DNA
ctcagggctgagatgccg

SEQ ID NO: 7 - DNA, 20 bp, synthetic construct
  source 1..20: mol_type = other DNA
tgtaaacatgacttccaagc

SEQ ID NO: 8 - DNA, 18 bp, synthetic construct
  source 1..18: mol_type = other DNA
aaaactgcaccttcacac

SEQ ID NO: 9 - DNA, 20 bp, synthetic construct
  source 1..20: mol_type = other DNA
gacaagcctgtagcccatgt

SEQ ID NO: 10 - DNA, 18 bp, synthetic construct
  source 1..18: mol_type = other DNA
tctcagctccacgccatt

SEQ ID NO: 11 - DNA, 20 bp, synthetic construct
  source 1..20: mol_type = other DNA
gatgtagtttaatccgacta

SEQ ID NO: 12 - DNA, 20 bp, synthetic construct
  source 1..20: mol_type = other DNA
taaaaatggaagaagcccaa

SEQ ID NO: 13 - DNA, 20 bp, synthetic construct
  source 1..20: mol_type = other DNA
tgcatcagagaacagatcct

SEQ ID NO: 14 - DNA, 20 bp, synthetic construct
  source 1..20: mol_type = other DNA
ctgggtaagaatgtaactcc

SEQ ID NO: 15 - protein, 342 aa, Homo sapiens
MDLWNWDEASPQEVPPGNKLAGLEGAKLGFCFPDLALQGDTPTATAETCWKGTSSSLASFPQLDWGSALLHPEVPWGAEPDSQALPWSGDWTDMACTAWDSWSGASQTLGPAPLGPGPIPAAGSEGAAGQNCVPVAGEATSWSRAQAAGSNTSWDCSVGPDGDTYWGSGLGGEPRTDCTISWGGPAGPDCTTSWNPGLHAGGTTSLKRYQSSALTVCSEPSPQSDRASLARCPKTNHRGPIQLWQFLLELLHDGARSSCIRWTGNSREFQLCDPKEVARLWGERKRKPGMNYEKLSRGLRYYYRRDIVRKSGGRKYTYRFGGRVPSLAYPDCAGGGRGAETQ